(19) United States
(12) Patent Application Publication
(10) Pub. No.: US 2024/0247290 A1
(43) Pub. Date: Jul. 25, 2024

(54) RECOMBINANT ALGAE HAVING HIGH LIPID PRODUCTIVITY

(71) Applicant: Viridos, Inc., La Jolla, CA (US)

(72) Inventors: Saheed Imam, La Jolla, CA (US); Eric R. Moellering, La Jolla, CA (US); Luke Peach, La Jolla, CA (US); Wiilliam F. Lambert, La Jolla, CA (US); Kathleen Kwok, La Jolla, CA (US)

(21) Appl. No.: 18/632,080

(22) Filed: Apr. 10, 2024

Related U.S. Application Data
(63) Continuation of application No. 17/519,383, filed on Nov. 4, 2021, now Pat. No. 11,976,315.
(60) Provisional application No. 63/110,301, filed on Nov. 5, 2020.

(51) Int. Cl. C12P 7/64; C07K 14/405; C12N 1/12

(52) U.S. Cl. CPC C12P 7/64; C07K 14/405; C12N 1/125

(57) ABSTRACT

The invention provides recombinant algal mutants that have a genetic modification to a gene or nucleic acid sequence encoding a WD40 repeat containing protein or domain. The genetic modification of one or more nucleic acid sequences encoding a WD40 repeat containing protein or domain results in a mutant organism with increased lipid productivity and/or higher biomass productivity (as measured by total organic carbon). The genetic modification can be a gene attenuation or functional deletion. The lipid products of these mutants can be utilized as biofuels or for other specialty chemical products. Methods of making and using the recombinant algal mutants and methods of producing lipids are also disclosed.

### CROSS-REFERENCE TO RELATED APPLICATIONS

This application is a continuation application of U.S. application Ser. No. 17/519,383 filed Nov. 4, 2021, now pending; which claims the benefit under 35 USC § 119(e) to U.S. Application Ser. No. 63/110,301 filed Nov. 5, 2020, now expired. The disclosure of each of the prior applications is considered part of and is incorporated by reference in the disclosure of this application.

### INCORPORATION BY REFERENCE OF SEQUENCE LISTING

The material in the accompanying sequence listing is hereby incorporated by reference into this application. The accompanying sequence listing xml file, named SGI2290-2_ST26.xml, was created on Mar. 29, 2024 and is 73,099 bytes in size.

### BACKGROUND OF THE INVENTION

### Field of the Invention

The invention involves the provision of a recombinant algal mutant organism and methods for the production of lipids.

### Background Information

The production of biofuels presents great opportunities to develop environmentally sound sources of energy that can be obtained at reasonable cost. Efforts have been directed towards using algae or other microorganisms to produce hydrocarbons that can be used as biodiesel or other biofuels due to their high lipid content. Additional specialty chemicals can also be obtained from these organisms and for use in consumer products.

Since algae use energy from sunlight to combine water and carbon dioxide to produce biomass, achieving increased productivity offers the possibility of a carbon neutral fuel source. The development of algal strains with very high lipid productivity for the production of algal-sourced biofuels therefore presents the possibility of a significant reduction in new carbon dioxide released into the atmosphere and a consequent reduction in the problem of global warming.

The development of commercially viable algal biofuels requires strains with high lipid and biomass productivity. Even the most productive wild type strains are not sufficiently productive to permit an economically viable development of this resource. Strategies for increasing algal production of biofuels and other products have included modification of nutrition provided to the organisms, such as cultivating the organisms in nitrogen, phosphorus, or silicon deficient media. Other strategies have included modification of cultivation conditions or environmental protocols, or various efforts directed towards genetic engineering of the organisms. While engineering algae strains to have a combination of increased photosynthetic efficiency (resulting in increased overall biomass productivity) and/or high lipid productivity could provide a solution to this problem, deficiencies still remain. The development of more productive strains continues to be a barrier to efficient utilization of this energy source.

### SUMMARY OF THE INVENTION

The invention provides recombinant algal mutants that have a genetic modification to a gene or nucleic acid sequence encoding a WD40 repeat containing protein. The genetic modification of one or more nucleic acid sequences encoding a WD40 repeat containing protein results in a mutant organism with increased lipid productivity and/or higher biomass productivity (as measured by total organic carbon). In some embodiments the genetic modification is a gene attenuation, or a deletion, disruption, or inactivation. The lipid products of these mutants can be utilized as biofuels or for other specialty chemical products. Methods of making and using the recombinant algal mutants and methods of producing lipids are also disclosed.

In a first aspect the invention provides a recombinant algal organism having a genetic modification in a gene or nucleic acid sequence encoding a WD40 repeat containing protein. The recombinant alga exhibits higher lipid productivity and/or higher biomass productivity versus a corresponding control algal cell not having the genetic modification. In one embodiment the recombinant alga is a Chlorophyte alga, which optionally can be of the Class Trebouxiophyceae. The gene or nucleic acid sequence encoding the WD40 repeat containing protein can have a nucleic acid sequence having at least 75% sequence identity to a nucleic acid sequence of any one of SEQ ID NOs: 14-26. In one embodiment the gene encoding the WD40 repeat containing protein has a nucleic acid sequence having at least 75% sequence identity to a nucleic acid sequence of SEQ ID NO: 15 or 16.

In one embodiment the genetic modification to the gene encoding the WD40 repeat containing protein is a functional deletion. The genetic modification of the gene encoding the WD40 repeat containing protein can result in an attenuation in the expression of the nucleic acid sequence. In one embodiment the genetic modification of the gene encoding the WD40 repeat containing protein is a deletion of one or more amino acids in a polypeptide sequence of a WD40 repeat containing protein. In one embodiment the genetic modification of the gene encoding the WD repeat containing protein is a frame shift mutation. The genetic modification can also be a deletion, a disruption, or an inactivation.

In one embodiment the recombinant alga has at least 30% higher lipid productivity versus a corresponding control alga that does not comprise the genetic modification. The recombinant alga can have at least 15% higher biomass productivity as measured by total organic carbon versus a control alga that does not comprise the genetic modification. The recombinant alga can exhibit at least 40 grams per square meter per day of lipid production after 5 days of cultivation.

In various embodiments the recombinant alga can higher biomass productivity per unit time as measured by production of total organic carbon (TOC). The recombinant alga can have higher biomass productivity under nitrogen deficient conditions. The recombinant alga can have higher total organic carbon production under nitrogen deficient conditions. In some embodiments the recombinant alga is of a family selected from Oocystaceae, Chlorellaceae, and Eustigmatophyceae; and the recombinant alga can be of a genus selected from Chlorella, Parachlorella, Picochlorum, Tetraselmis, and Oocystis.

In another aspect the invention provides a lipid containing product produced by any of the recombinant algae described herein.

In another aspect the invention provides a biomass product containing any of the recombinant algae described herein.

In another aspect the invention provides a method of producing a composition containing lipids. The methods involve cultivating a recombinant alga described herein and thereby producing a composition containing lipids. The method can also involve harvesting a lipidic composition from the recombinant alga. The method can involve producing a genetic modifications to a nucleic acid sequence encoding a WD repeat protein, which can be a functional deletion. The functional deletion can be obtained by subjecting the algal organism to ultra-violet light. In some embodiments the gene encoding the WD repeat containing protein has a nucleic acid sequence having at least 90% sequence identity to SEQ ID NO: 16. The algal organism can be a Chlorophyte alga, optionally of the genus Oocystis, but can be any recombinant alga described herein. The recombinant alga produced by the method can have at least 30% greater lipid productivity versus a control alga.

In another aspect the invention provides a method of producing a composition containing lipids. The methods involve performing a genetic modification to an algal organism in a gene encoding a WD40 repeat containing protein, and culturing the organism to thereby produce a composition containing lipids. The method can further involve harvesting a lipidic composition from the algal organism.

In another aspect the invention provides a method for identifying a recombinant algal organism with high lipid productivity and/or high biomass productivity. The method involves mutagenizing a population of algal organisms; screening the mutagenized algal organisms for higher lipid productivity; sequencing at least a portion of the genome of the mutagenized algal organisms; identifying genetic changes in the mutagenized organisms compared to the population of algal organisms prior to mutagenesis; recapitulating the genetic changes in a parental strain of the mutagenized algal organisms; and thereby identifying a recombinant algal organism having high lipid productivity. The method can further involve harvesting a lipidic composition from the recombinant algal organism. The recombinant algal organism with high lipid productivity and/or high biomass productivity can be any recombinant algal organism described herein. In one embodiment the recombinant algal organism can have at least 40% greater lipid productivity versus a control alga.

The summary of the invention described above is not limiting and other features and advantages of the invention will be apparent from the following detailed description of the invention, and from the claims. Section headings or sub-headings are provided solely for the convenience of the reader, and do not denote a departure from discussion or necessarily an entirely new subject matter area. Any subject matter can be discussed or disclosed under any section heading or sub-heading.

### DETAILED DESCRIPTION OF THE DRAWINGS

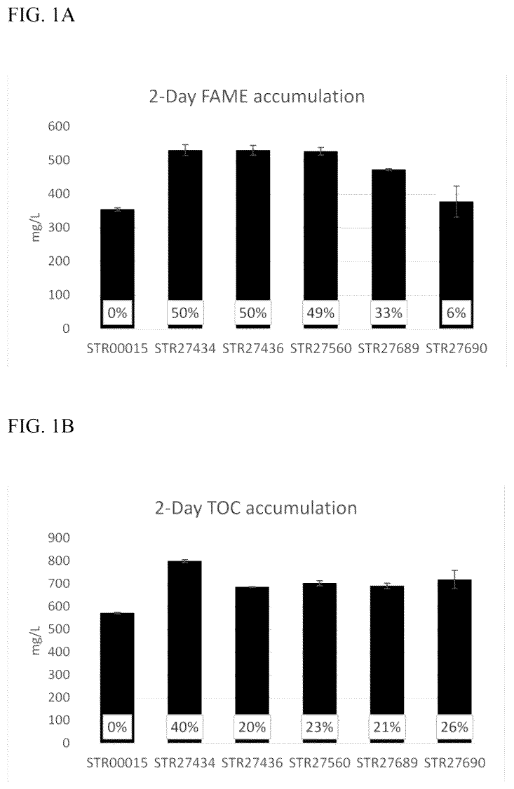

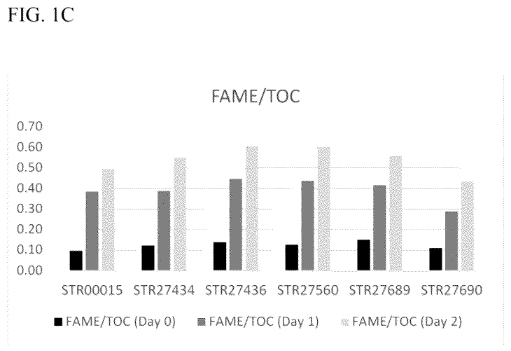

FIGS. 1A-1C are graphical illustrations of the effects on FAME and TOC accumulation in strains having a genetic modification to a nucleic acid sequence encoding a WD40 repeat family protein by mutagenesis of wild type cells (Strain 15). FIG. 1A shows 2-day FAME accumulation for various Oocystis strains. FIG. 1B shows 2-day TOC accumulation. FIG. 1C shows the ratio of FAME/TOC for various strains as an indicator of carbon partitioning. Mutagenized strains include 27434, 27436, 27550, 27689, and 27690, which all contain a deletion to SEQ ID NO: 1 (a WD40 repeat containing protein). The 2-day data was measured after 48 hours of growth in nitrogen deplete media.

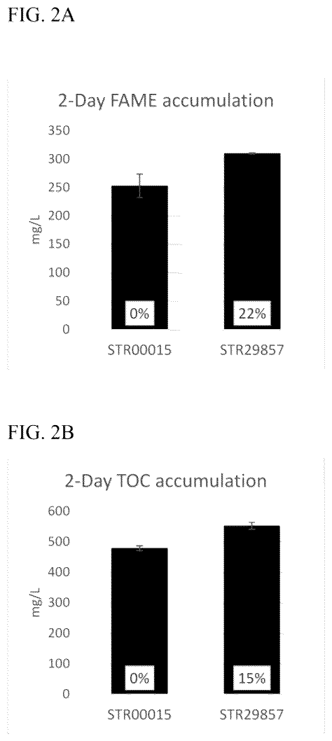

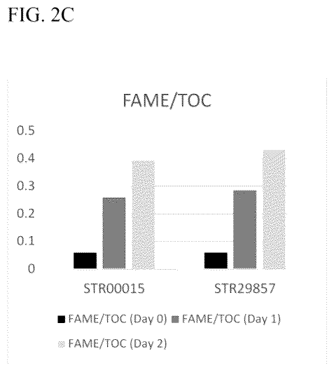

FIGS. 2A-2C are graphical illustrations of the FAME and TOC accumulation in wild type strains constructed to have a deletion of a nucleic acid sequence encoding a WD40 repeat family protein. FIG. 2A shows 2-day FAME accumulation for modified Oocystis sp. strains (29857) versus the wild type (Strain 15). FIG. 2B shows 2-day TOC accumulation. FIG. 2C shows the ratio of FAME/TOC, a useful measure of carbon partitioning.

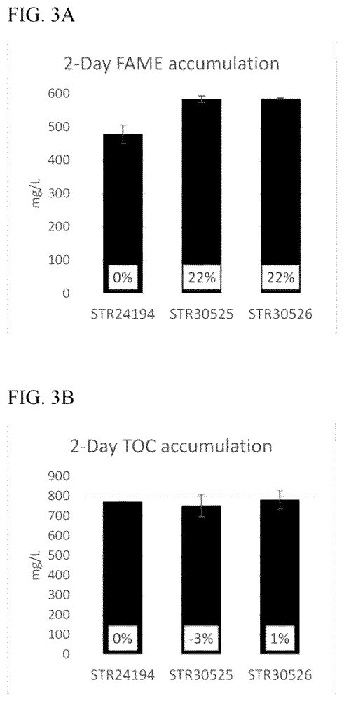

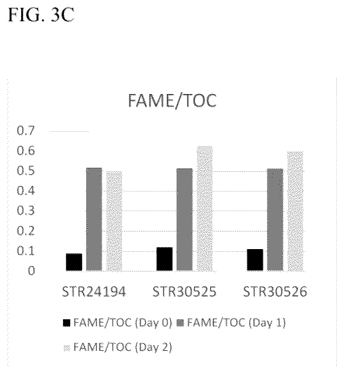

FIGS. 3A-3C are graphical illustrations of the FAME and TOC accumulation in a laboratory strain (24194) constructed to have a deletion of a nucleic acid sequence encoding a WD40 repeat family protein versus the (control) laboratory strain. FIG. 3A shows 2-day FAME accumulation for the laboratory strain (Strain 24194) and modified Oocystis sp. strains (30525 and 30526). FIG. 3B shows 2-day TOC accumulation. FIG. 3C shows the ratio of FAME/TOC, a useful measure of carbon partitioning.

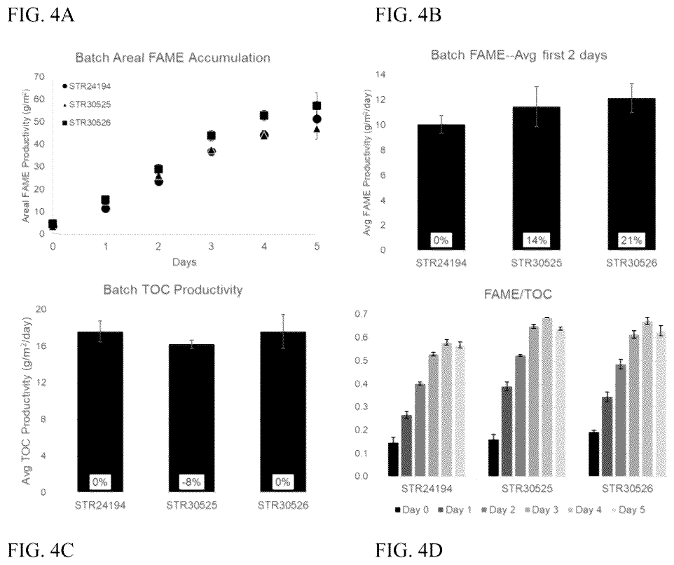

FIGS. 4A-4D provide a graphical illustration of 5-day data for FAME and TOC productivity under nitrogen deplete conditions. FIG. 4A shows areal FAME productivity v. days. FIG. 4B shows average FAME productivity for the first 2 days. FIG. 4C shows TOC productivity. And FIG. 4D shows the FAME/TOC ratios over a 5 day period.

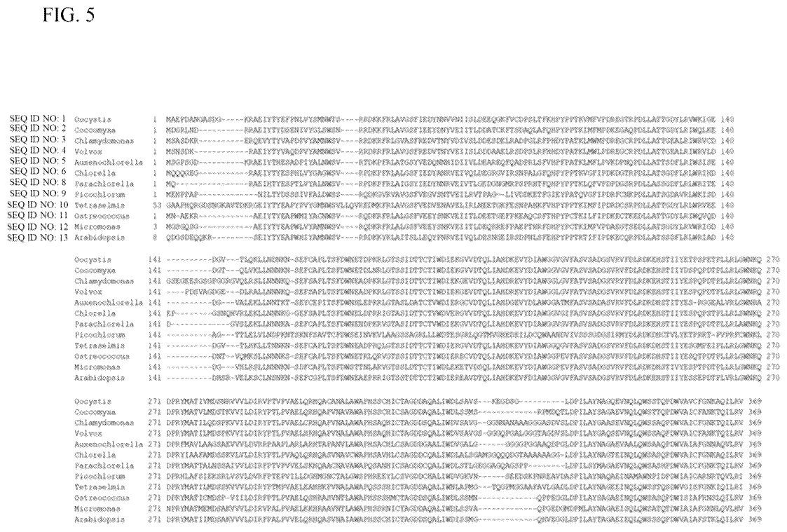

FIG. 5 is a sequence alignment of protein sequences of various Chlorophyte algae species. It is shown that there is a high degree of sequence identity across Chlorophyte species in key domains of the polypeptide sequence.

### DETAILED DESCRIPTION OF THE INVENTION

The invention provides recombinant algal mutants that have a genetic modification to a nucleic acid sequence encoding a WD40 repeat containing protein. The genetic modification(s) described herein result in a recombinant or mutant cell or organism with higher lipid productivity and/or higher biomass productivity. The recombinant algal mutants can also optionally have reduced chlorophyll content and/or a reduced PSII antenna size versus a corresponding control cell or organism not having the genetic modification. In various embodiments the genetic modification(s) described herein can result in substantial increases in lipid productivity and/or biomass productivity. Any of the recombinant cells or organisms disclosed herein can be mutant photosynthetic organisms.

The recombinant cell or organism of the invention having a genetic modification described herein can have higher lipid productivity and/or higher biomass productivity than a corresponding (control) cell or organism. In some embodiments the genetic modification is an attenuation of a gene or nucleic acid sequence encoding a WD40 repeat containing protein. In any embodiment lipid productivity can be measured by FAME accumulation, and biomass productivity can be measured as the rate of biomass accumulation, for example as the total organic carbon (TOC) content of the respective cells or organisms.

In one embodiment the mutant or recombinant algal cell or organism has higher lipid and/or biomass productivity in batch culture compared to a corresponding (control) cell or organism not having the genetic modification. Batch culture is a culture where nutrients are not renewed or re-supplied to the medium during the time period the cells or organisms are cultured. In other embodiments the mutant or recombinant algal cell or organism has higher lipid and/or biomass productivity under nitrogen deplete conditions compared to a corresponding (control) cell or organism. In some embodiments the higher lipid and/or biomass productivity is achieved under batch culture and nitrogen deplete conditions; and in other embodiments the higher lipid and/or biomass productivity is achieved under semi-continuous or continuous culture conditions. Semi-continuous culture conditions are conditions where a fixed volume of culture is removed at regular time intervals and an equal volume of fresh medium is immediately added back to the culture. In various embodiments the fixed volume removed and added is from 10% to 90% of the total culture volume. Continuous culture conditions involve regular removal of culture with immediate replacement with fresh medium. Persons of ordinary skill realize that time intervals can vary with the purpose of the culture and continuous culture conditions involve a more frequent removal of culture and replacement with fresh medium than semi-continuous conditions.

Any of the mutant or recombinant cells or organisms disclosed herein can be photosynthetic cells or organisms. Any of the mutant or recombinant cells or organisms described herein can exhibit increased lipid productivity and/or increased biomass productivity under photoautotrophic conditions compared to a corresponding control cell or organism, i.e., conditions where the recombinant cells or organisms can produce their own biomass using light, carbon dioxide, water, and nutrients via photosynthesis. Corresponding (control) cells or organisms are useful for evaluating the effect of any one or more of the genetic modifications. Corresponding (control) cells or organisms do not have the one or more genetic modifications being evaluated and are subjected to the same or substantially the same conditions as the test cells or organisms such that a difference in the performance or characteristics of the cells or organisms is based only on the genetic modification(s) being evaluated. In any embodiment the corresponding (control) cells or organisms can be of the same species as the test organism. They can be derived from the same parent cell or parental line. They can also be the same or similar in every way except for the one or more genetic modification(s) being evaluated. In some embodiments the corresponding (control) cell or organism is a wild-type cell or organism. But the corresponding (control) cell or organism can also be a laboratory strain or parental strain of the test cell or organism. Substantially the same conditions can be the same conditions or slightly different conditions where the difference does not materially affect the function, activity, or expression of the nucleic acid sequence modified.

In various embodiments the recombinant cells or organisms are algal cells. In one embodiment the recombinant alga has a genetic modification to a gene encoding a WD40 repeat containing protein. The lipid products of these mutants can be further processed into biofuels or used in the production of other specialty chemical products. The gene or nucleic acid sequence encoding the WD40 repeat containing protein or domain that contains the genetic modification can encode any of the polypeptide sequences described herein, hereby disclosed in all possible combinations or sub-combinations as if set forth fully herein.

In some embodiments recombinant cells or organisms of the invention can have a reduced amount of chlorophyll b, and can have an increased chlorophyll a to chlorophyll b ratio (chl a/chl b) compared to a corresponding control cell or organism. The recombinant cells or organisms can have decreased photosynthetic antenna size, for example reduced photosystem II (PSII) and/or reduced photosystem I (PSI) antenna size. In various embodiments the cross-sectional unit size of the PSII and/or PSI antenna of the recombinant cells or organisms disclosed herein can be reduced by at least 10%, at least 20%, at least 30%, at least 25%, at least 30%, at least 35%, at least 40%, at least 45%, at least 50%, or at least 60% compared to the PSII and/or PSI antenna size of a corresponding control cell or organism.

In any embodiment the recombinant cells or organisms can have a higher growth rate and/or a higher biomass productivity than a corresponding control cell or organism not having the genetic modification, for example, higher biomass productivity per hour or per day or per period of 2 days or 3 days or 4 days or 5 days or 6 days. “Biomass” refers to cellular mass, whether of living or dead cells. Biomass productivity, or biomass accumulation, or growth rate, can be measured by any means accepted in the art, for example as ash free dry weight (AFDW), dry weight, wet weight, or total organic carbon (TOC) productivity. In any embodiment biomass productivity, or biomass accumulation, or the growth rate, can be measured as total organic carbon (TOC) productivity.

The recombinant cells or organisms of the invention can produce a greater amount of a bioproduct per time period (e.g., per minute or per hour or per day or per period of 2 days or 3 days or 4 days or 5 days or 6 days), for example a lipid product (which can optionally be measured as FAME), a fatty acid product, a carbohydrate, a protein product, or a polymer than a corresponding (control) organism not having the genetic modification(s). The amount of bioproduct can be expressed as g/time period, mg/time period, ug/time period, or any other defined quantity per defined time period described herein. Such bioproducts can be isolated from a lysate or biomass or cellular secretion of any of the recombinant cells or organisms of the invention. In some embodiments, the recombinant cells or organisms of the invention produce at least 10%, at least 15%, at least 20%, at least 25%, at least 30%, at least 35%, at least 40%, at least 45%, at least 50%, at least 60%, at least 70%, at least 80%, at least 90% or at least 100% more of a bioproduct than a corresponding control alga cultured under the substantially the same conditions, which can be batch, semi-continuous, or continuous culture conditions and may be nutrient replete culture conditions or may be nitrogen deplete conditions, and may be photoautotrophic conditions. In some embodiments the recombinant cells or organisms of the invention product at least 20% or at least 30% more lipid (e.g., as measured by FAME) over a 2-day period, or at least 30% or at least 40% more of a lipid product over a 5-day period.

Without wanting to be bound by any particular theory it is believed that the genetic modifications described herein result in an attenuation or partial or complete elimination of expression of a WD40 repeat containing protein. Such attenuation or elimination results in a significant increase in lipid productivity in the cell, which in one embodiment can be measured as the total FAME produced by the cell. A further result can be a significant increase in biomass productivity, which in one embodiment can be demonstrated by the organic carbon produced by the cell (as measured, for example, by total organic carbon).

As used herein, “exogenous” with respect to a nucleic acid or gene indicates that the nucleic acid or gene has been introduced (e.g., “transformed”) into an organism, microorganism, or cell by human intervention. For example, such an exogenous nucleic acid can be introduced into a cell or organism via a recombinant nucleic acid construct. An exogenous nucleic acid can be a sequence from one species introduced into another species, i.e., a “heterologous” nucleic acid. A heterologous nucleic acid can also be an exogenous synthetic sequence not found in the species into which it is introduced. An exogenous nucleic acid can also be a sequence that is homologous to an organism (i.e., the nucleic acid sequence occurs naturally in that species or encodes a polypeptide that occurs naturally in the host species) that has been isolated and subsequently reintroduced into cells of that organism. In some embodiments an exogenous nucleic acid that includes a homologous sequence can be distinguished from the naturally-occurring sequence by the presence of non-natural sequences linked to the exogenous nucleic acid, which can include but are not limited to non-native regulatory sequences attached to the homologous gene sequence in a recombinant nucleic acid construct. Alternatively or in addition, a stably transformed exogenous nucleic acid can be detected and/or distinguished from a native gene by its juxtaposition to sequences in the genome where it has integrated. Further, a nucleic acid is considered exogenous if it has been introduced into a progenitor of the cell, organism, or strain under consideration.

A “recombinant” or “engineered” nucleic acid molecule is a nucleic acid molecule that has been altered through human manipulation. As non-limiting examples, a recombinant nucleic acid molecule includes any nucleic acid molecule that: 1) has been partially or fully synthesized or modified in vitro, for example, using chemical or enzymatic techniques (e.g., by use of chemical nucleic acid synthesis, or by use of enzymes for the replication, polymerization, digestion (exonucleolytic or endonucleolytic), ligation, reverse transcription, transcription, base modification (including, e.g., methylation), integration or recombination (including homologous and site-specific recombination) of nucleic acid molecules); 2) includes conjoined nucleotide sequences that are not conjoined in Nature; 3) has been engineered using molecular biology techniques such that it lacks one or more nucleotides with respect to the naturally occurring nucleic acid molecule sequence; and/or 4) has been manipulated using molecular biology techniques such that it has one or more sequence changes or rearrangements with respect to the naturally occurring nucleic acid sequence, or has a sequence (e.g., by insertion) not found in the naturally occurring nucleic acid sequence. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector.

When applied to organisms, the terms “transgenic” “transformed” or “recombinant” or “engineered” or “genetically engineered” refer to organisms that have been manipulated by introduction of an exogenous or recombinant nucleic acid sequence into the organism, or by genetic modification of native sequences (which are therefore then recombinant). Recombinant or genetically engineered organisms can also be organisms into which constructs for gene “knock down,” deletion, attenuation, inactivation, or disruption have been introduced to perform the indicated manipulation. Such constructs include, but are not limited to, RNAi, microRNA, shRNA, antisense, and ribozyme constructs. A recombinant organism can also include those having an introduced exogenous regulatory sequence operably linked to an endogenous gene of the transgenic microorganism, which can enable transcription in the organism. Also included are organisms whose genomes have been altered by the activity of meganucleases or zinc finger nucleases. A heterologous or recombinant nucleic acid molecule can be integrated into a genetically engineered/recombinant organism's genome or, in other instances, not integrated into a recombinant/genetically engineered organism's genome, or can be present on a vector or other nucleic acid construct. As used herein, “recombinant microorganism” or “recombinant host cell” includes progeny or derivatives of the recombinant microorganisms of the disclosure.

Any of the recombinant algal cells or organisms described herein can be generated by human activity, for example, by classical mutagenesis and/or genetic engineering, but can also be produced by any feasible mutagenesis method, including but not limited to exposure to UV light, CRISPR/Cas9, cre/lox, gamma irradiation, or chemical mutagenesis. Screening methods can be used to identify mutants having desirable characteristics (e.g., reduced chlorophyll and increased lipid and/or biomass productivity). Methods for generating mutants of algal organisms using classical mutagenesis, genetic engineering, and phenotype or genotype screening are known in the art.

### Algal Cell or Organism

The recombinant algal cell or organism of the invention can be a mutant microalga, or a mutant photosynthetic organism, or a mutant green alga. The recombinant alga can be any eukaryotic microalga such as, but not limited to, a Chlorophyte, an Ochrophyte, or a Charophyte alga. In some embodiments the mutant microalga can be a Chlorophyte alga of the taxonomic Class Chlorophyceace, or of the Class Chlorodendrophyceae, or the Class Prasinophyceace, or the Class Trebouxiophyceae, or the Class Eustigmatophyceae. In some embodiments, the mutant microalga can be a member of the Class Chlorophyceace, such as a species of any one or more of the genera Asteromonas, Ankistrodesmus, Carteria, Chlamydomonas, Chlorococcum, Chlorogonium, Chrysosphaera, Dunaliella, Haematococcus, Monoraphidium, Neochloris, Oedogonium, Pelagomonas, Pleurococcus, Pyrobotrys, Scenedesmus, or Volvox. In other embodiments the mutant microalga of the invention can be a member of the Order Chlorodendrales, or Chlorellales. In other embodiments, the mutant microalga can be a member of the Class Chlorodendrophyceae, such as a species of any one or more of the genera Prasinocladus, Scherffelia, or Tetraselmis. In further alternative embodiments, the mutant alga can be a member of the Class Prasinophyceace, optionally a species of any one or more of the genera Ostreococcus or Micromonas. Further alternatively, the mutant microalga can be a member of the Class Trebouxiophyceae, and optionally of the Order Chlorellales, and optionally a genera selected from any one or more of Botryococcus, Chlorella, Auxenochlorella, Heveochlorella, Marinichlorella, Oocystis, Parachlorella, Pseudochlorella, Tetrachlorella, Eremosphaera, Franceia, Micractinium, Nannochloris, Picochlorum, Prototheca, Stichococcus, or Viridiella, or any of all possible combinations or sub-combination of the genera. In another embodiment the recombinant alga can be a Chlorophyte alga of the Class Trebouxiophyceae and the family Coccomyxaceae, and the genus Coccomyxa (e.g., Coccomyxa subellipsoidea). Or of the family Chlamydomonadaceae and the genus Chlamydomonas (e.g., Chlamydomonas reinhardtii); or of the family Volvocaceae and the genus Volvox (e.g., Volvox carteri, Volvox aureus, Volvox globator).

In another embodiment the recombinant alga is a Chlorophyte alga of the Class Trebouxiophyceae, or Eustigmatophyceae, and can be of the Order Chlorellales or Chlorodendrales, and can be of the Family Oocystaceae, or Chlorellaceae, or Monodopsidaceae, and optionally from a genus selected from any one or more of Oocystis, Parachlorella, Picochlorum, Nannochloropsis, and Tetraselmis, in all possible combinations and sub-combinations. The recombinant alga can also be from the genus Oocystis, or the genus Parachlorella, or the genus Picochlorum, or the genus Tetraselmis, or from any of all possible combinations and sub-combinations of the genera. In one embodiment the recombinant algal cell or organism is of the Class Trebouxiophyceae, of the Order Chlorellales, and optionally of the family Oocystaceae, and optionally can be of the genus Oocystis.

### Genetic Modification

In various embodiments the recombinant alga of the invention can have a genetic modification disclosed herein to a gene encoding a WD40 repeat containing protein. In one embodiment the recombinant alga of the invention has a genetic modification to a gene encoding a WD40 repeat containing protein or domain. In one embodiment the genetic modification is to a native or endogenous sequence of the cell or organism.

A “genetic modification” applied in the invention can be any one or more of an attenuation, a deletion, a gene “knock out,” a mutation, a disruption, an insertion, insertion of a stop codon, an inactivation, a rearrangement, one or more point mutations, a frameshift mutation, a nonsense mutation, an inversion, a single nucleotide polymorphism (SNP), a truncation, a point mutation, that changes the activity or expression of the one or more gene or nucleic acids. In some embodiments the change in expression is a reduction in expression or an elimination of the expression or activity, which expression can be the production of an encoded protein. The genetic modification can be made or be present in any sequence that affects expression or activity of the gene or nucleic acid sequence, or the nature or quantity of its product, for example to a coding or non-coding sequence, a promoter, a terminator, an exon, an intron, a 3′ or 5′ UTR, or other regulatory sequence. A genetic modification can be performed in any structure of the gene that results in attenuation or elimination of the gene or nucleic acid product or activity. In one embodiment the genetic modification is a deletion, disruption, or inactivation. The genetic modification can be made to, or be present in, the host cell's native genome. In some embodiments, a recombinant cell or organism having attenuated expression of a gene as disclosed herein can have one or more mutations, which can be one or more nucleobase changes and/or one or more nucleobase deletions and/or one or more nucleobase insertions, into the region of a gene 5′ of the transcriptional start site, such as, in non-limiting examples, within about 2 kb, within about 1.5 kb, within about 1 kb, or within about 0.5 kb of the known or putative transcriptional start site, or within about 3 kb, within about 2.5 kb, within about 2 kb, within about 1.5 kb, within about 1 kb, or within about 0.5 kb of the translational start site. The genetic modification can comprise the deletion of one or more amino acids in a polypeptide sequence of a WD40 repeat containing protein.

A frameshift mutation is a particular type of mutation that involves either insertion or deletion of bases of DNA, where the number of inserted or deleted bases is not divisible by three. As a result a frameshift mutation disrupts the natural codon reading frame. This can result in insertion of an amino acid other than the naturally occurring programmed amino acid. Some or all of the entire DNA sequence following the mutation can also be disrupted or read incorrectly, i.e., as programming an amino acid other than the naturally programmed amino acid. In some embodiments a frame shift mutation can result in premature termination of translation. Thus, in such embodiments the encoded polypeptide sequence can be much shorter, and unable to accomplish its natural function in the cell or organism. Thus, the gene or nucleic acid sequence has been functionally deleted. A nonsense mutation is the substitution of one or more base pair(s) that leads to a stop codon being inserted into a nucleic acid sequence. As a result, a nonsense mutation results in premature termination of translation, and can mean the functional deletion of the gene or nucleic acid sequence.

An “attenuation” is a genetic modification resulting in a reduction of the function, activity, or expression of a gene or nucleic acid sequence compared to a corresponding (control) cell or organism not having the genetic modification being examined, i.e., the diminished function, activity, or expression is due to the genetic modification. The activity of a nucleic acid sequence can be expression of an encoded polypeptide, a binding activity, the amount of signal transduction or transcription regulation, or other activity the nucleic acid sequence exerts within the organism. In various embodiments an attenuated gene or nucleic acid sequence disclosed herein produces less than 90%, or less than 80%, or less than 70%, or less than 50%, or less than 30%, or less than 20%, or less than 10%, or less than 5% or less than 1% of its function, activity, or expression of the gene or nucleic acid sequence compared to the corresponding (control) cell or organism. In various embodiments a gene attenuation can be achieved via a deletion, a disruption, or an inactivation. Any of the genetic modifications described herein can result in partial or complete attenuation of the function, activity, or expression of the attenuated gene or nucleic acid sequence, which in some embodiments can lead to a level of function, activity, or expression that is not substantially more than that of complete attenuation; for example, the function, activity, or expression can yield a result that is less than 10% different from a recombinant cell or organism having a complete attenuation of the gene or nucleic acid sequence.

An unmodified gene or nucleic acid sequence present naturally in the organism denotes a natural, endogenous, or wild type sequence. A deletion can mean that at least part of the object nucleic acid sequence is deleted or that at least part of the encoded product is eliminated. But a deletion can also be accomplished by disrupting a gene through, for example, the insertion of a sequence into the gene that is not naturally present (e.g., a selection marker), a combination of deletion and insertion, or mutagenesis resulting in insertion of a stop codon. But a deletion can also be performed by other genetic modifications known to those of ordinary skill that result in the loss of expression, activity, or function of a gene or nucleic acid sequence.

A functional deletion is a genetic modification that removes at least so much of the activity or expression of a gene or nucleic acid sequence so that any remaining activity or expression of the gene or nucleic acid sequence has no significant effect on the cell or organism compared to a corresponding (control) cell or organism not having the functional deletion and cultivated under the same or substantially the same conditions. In some embodiments the functional deletion can remove all function, activity, or expression of the gene or nucleic acid sequence. A functional deletion can involve an at least partial deletion of the coding or non-coding sequence of the gene. A “deletion” or “knock out” removes all function, activity, or expression of a gene or nucleic acid sequence. A “disruption” of a gene is a functional deletion by insertion or deletion of a nucleotide sequence into or from the coding, non-coding, or regulatory portion of a gene with resulting partial or complete loss of function, activity, or expression of the gene. An “inactivation” is a type of functional deletion causing partial or complete loss of activity or expression of an inactivated gene or nucleic acid sequence. An “inactivation” can be reversible or irreversible (for example the reversible or irreversible binding of a component to the gene or nucleic acid sequence). Thus, deletions, functional deletions, inactivations, and disruptions can also be attenuations; disruptions and inactivations can also be functional, i.e., can remove at least so much of the activity or expression of a gene or nucleic acid sequence so that any remaining activity or expression of the gene or nucleic acid sequence has no significant effect on the cell or organism compared to a corresponding (control) cell or organism. An attenuation can also be a downregulation of a gene or nucleic acid sequence, which refers to the cell or organism decreasing the amount of function, activity, or expression. Functional expression refers to the expression of a functional product or activity of a nucleic acid sequence. When the expressed product of a nucleic acid is a polypeptide, functional expression means expression of polypeptide activity having at least 10% or at least 25% or at least 50% or at least 75% of the activity of a corresponding (control) cell or organism not having the modification and cultivated under the same or substantially the same conditions. For activity of a gene or nucleic acid sequence functional expression means activity or expression of at least 10% or at least 25% or at least 50% or at least 75% of the activity or expression of a corresponding (control) cell or organism not having the modification at issue and cultivated under the same or substantially the same conditions. Thus, various types of genetic modifications can be given terms that overlap in description. Persons of ordinary skill know that the particular term describing a genetic modification can be dependent both on how a gene or its components, or nucleic acid sequence is being physically changed as well as on the context. The recombinant cells or organisms of the invention can have any of the types of genetic modifications described herein.

In one embodiment the genetic modification is a deletion (or “knock out”) involving the introduction of a stop codon into a gene or nucleic acid sequence encoding a WD40 repeat containing protein described herein or variant thereof. For example the genetic modification can be a stop codon or frame shift mutation (or other genetic modification described herein) introduced into a gene or nucleic acid of any one of SEQ ID NO: 14-26 or a variant of any, or in a gene or nucleic acid sequence encoding any one of SEQ ID NOs: 1-13 (WD40 repeat containing proteins), or a variant of any. In one embodiment the genetic modification is a stop codon or frame shift mutation inserted into a gene or nucleic acid sequence of SEQ ID NO: 15 or 16 or a variant of either, or encoding SEQ ID NO: 1, or a variant thereof. In another embodiment the genetic modification is a stop codon or frame shift mutation inserted into a gene or nucleic acid sequence encoding SEQ ID NO: 8, or a variant thereof (WD40 repeat containing protein of Parachlorella). Variant sequences of any sequence described herein have at least 60% sequence identity or at least 70% sequence identity or at least 75% or at least 80% or at least 85% or at least 90% or at least 95% or at least 98% sequence identity to any nucleotide or polypeptide sequence of any of SEQ ID NOs: 1-26. In some embodiments a variant sequence can also have at least 25 or at least 50 or at least 75 or at least 100 or at least 150 or at least 200 or at least 250 or at least 300 or at least 350 or at least 400 contiguous amino acids of any one of SEQ ID NOs: 1-13, and optionally, also have a recited percent identity. In other embodiments the variants sequence can have at least 500 or at least 600 or at least 700 or at least 800 or at least 1000 or at least 1200 or at least 1400 or at least 1500 contiguous nucleotides of any sequence of SEQ ID NO: 14-26 and, optionally, also have a recited percent identity.

In one embodiment the genetic modification is a modification that results in a stop codon, nonsense mutation, or frame shift mutation (or other genetic modification described herein) at the sequence coding for SEQ ID NO: 14 (AGACTCGCACCG) (V204fs), or a variant thereof, or in SEQ ID NO: 15 or 16, or a variant of either. The genetic modification can also be targeted to a regulatory sequence with the effect of eliminating or diminishing the activity or expression of a gene or nucleic acid sequence of SEQ ID NO: 14-26 or a variant of any, or a gene or nucleic acid sequence that encodes for any one SEQ ID NO: 1-13 or a variant of any of them.

In some embodiments the genetic modification can also be a frame shift mutation, a stop codon mutation, or a nonsense mutation introduced into a gene or nucleic acid sequence (including regulatory sequences supporting such gene or nucleic acid sequences) encoding any WD40 repeat containing protein disclosed herein. In various embodiments the gene or nucleic acid sequence is of SEQ ID NO: 14-26, or a variant of any; in a specific embodiment the gene or nucleic acid sequence encodes for a polypeptide of SEQ ID NO: 1 (or a variant thereof) or a polypeptide of SEQ ID NO: 8 (or a variant thereof), which mutation can be introduced at any location of the sequence or into a regulatory sequence governing the sequence, where the modification results in a termination of transcription from the gene prior to its natural point. Thus, in one embodiment the mutation is the introduction of a frame shift mutation, stop codon, or nonsense mutation that functionally deletes or disrupts the activity or expression of the gene or nucleic acid sequence. The frame shift mutation, stop codon, or nonsense mutation (or other modification) can also be introduced at any of many different loci or locations within a gene encoding a WD40 repeat containing protein or in a regulatory sequence, for example at a promoter, terminator or other regulatory sequence, that attenuates the gene or the activity of the encoded polypeptide, and that results in functional deletion of the gene. Analogous modifications can be made to the sequence(s) for similar effect. Such insertion or deletion or other mutation can also cause a loss of expression, function or activity in the encoded WD40 repeat containing protein, and result in the effect of increased lipid productivity.

In other embodiments the genetic modification can be to a nucleic acid sequence of SEQ ID NO: 15 or 16, or to a variant of either having at least 70% sequence identity or at least 75% or at least 80% or at least 85% or at least 90% or at least 95% or at least 98% sequence identity to any nucleotide sequence of SEQ ID NOs: 15 or 16.

Any of the recombinant cells or organisms of the invention can have a reduced functional absorption cross section of PSII and/or reduced PSII antenna size. For example, the cross-sectional unit size of the PSII antenna can be reduced by at least about 10%, at least 20%, at least 30%, at least 25%, at least 30%, at least 35%, at least 40%, at least 45%, at least 50%, at least 60%, at least about 70%, or at least about 80% compared to the functional absorption cross section of PSII and/or PSII antenna size of the corresponding (control) cell or organism not having the genetic modification. In some embodiments the recombinant cells or organisms of the invention can additionally (and optionally) have a reduced functional absorption cross section of PSI or reduced PSI antenna size by the same amounts stated above versus a corresponding (control) cell or organism. All statements herein of increases in biochemical parameters (e.g., FAME productivity or TOC productivity) are versus a corresponding (control) cell or organism unless indicated otherwise.

In some embodiments, a recombinant algal cell or organism as provided herein can have increased Fv/Fm with respect to a corresponding control cell or organism. For example, the mutant photosynthetic organism may have Fv/Fm increased by at least 5%, at least 10%, at least 12%, at least 15%, at least 20%, at least 30%, at least 40% or at least 50% compared to a corresponding (control) photosynthetic organism. In various embodiments the Fv/Fm can be increased by 5-50%, or by 5-30% or by 5-20% with respect to a control photosynthetic organism.

Further, a mutant photosynthetic organism as provided herein can have an increased rate of electron transport on the acceptor side of photosystem II with respect to a control or wild type cell. The rate can be at least about 20%, 30%, 40%, 50%, 60%, 80%, or 100% higher compared to a corresponding control cell or organism. In addition, mutant photosynthetic cells or organisms of the invention can have a rate of carbon fixation (Pmax (C)) in a recombinant cell or organism as provided herein can be elevated with respect to a control organism. For example, Pmax (14 C) can be increased by at least about 20%, 30%, 40%, 50%, 60%, 80%, or 100% compared to a corresponding control cell or organism.

In some embodiments, the recombinant cells or organisms of the invention have decreased PSI and/or PSII antenna size and can optionally also have a higher amount of a ribulose bisphosphate carboxylase activase (Rubisco activase or “RA”) than a corresponding (control) or wild type organism, for example, at least 1.2, 1.4, 1.6, 1.8, 2, 2.2, or 2.5 fold the amount of RA as a control organism. In some embodiments, the mutants demonstrate reduced expression of 6, 8, 10, 12, or 14 LHCP genes and increased expression of an RA gene, such as an RA-a or RA-P gene. Thus, the recombinant cells or organisms of the invention can be mutant photosynthetic organisms having reduced chlorophyll and reduced PSII antenna size where the mutants have a higher amount of Rubisco activase than control photosynthetic organisms.

The LHC super-gene family encodes the light-harvesting chlorophyll a/b-binding (LHC) proteins that constitute the antenna system of the photosynthetic apparatus. A recombinant algal mutant of the invention can also have a reduced expression of one or more LHC genes. Thus, in some embodiments the recombinant cells or organisms of the invention have at least 6, at least 8, at least 10, or at least 12 LHC genes that are attenuated or downregulated with respect to their expression level in a corresponding (control) cell or organism. In various embodiments the reduction in expression of the one or more LHC genes can be a reduction of at least 20%, or at least 30%, or at least 40%, or at least 50%, or at least 60%, or at least 70% in the level of LHC transcripts compared to the control cell or organism.

The structure of a gene consists of many elements, of which the protein coding sequence is only one part. The gene includes nucleic acid sequences that are not transcribed and sequences that are untranslated regions of the RNA. Genes also contain regulatory sequences, which includes promoters, terminators, enhancers, silencers, introns, 3′ and 5′ UTRs, and coding sequences, as well as other sequences known to be a part of genes. In various embodiments any of these structures or nucleic acid sequences (e.g., any of SEQ ID NO: 14-26 or variants of any) can have one or more of the genetic modifications described herein that result in the higher lipid productivity and/or higher biomass productivity as described herein.

### WD40 Repeat Containing Protein

WD40 repeat containing proteins (or domains) have four or more repeating units containing a conserved core motif that ends with tryptophan-aspartic acid (WD). Proteins of this class have domain folds that form a beta propeller structure, which are symmetric folds having 4-9 anti-parallel, four-stranded beta sheets arranged radially around a central axis. The domains form a platform on which protein complexes assembly reversibly. They are believed to play a key role in the formation of protein-protein complexes. In any embodiment the genetic modification to the WD40 repeat containing protein can be to any WD40 repeat domain of the protein.

The recombinant algal cell or organism of the invention can have a genetic modification described herein to a nucleic acid sequence of any of SEQ ID NO: 14-26, or to a nucleic acid sequence having at least 60% sequence identity or at least 70% sequence identity or at least 75% or at least 80% or at least 85% or at least 90% or at least 95% or at least 98% sequence identity to any of SEQ ID NOS: 14-26. The nucleic acid sequence can encode a WD40 repeat containing protein. The genetic modification can also be to any of the disclosed sequences and also have at least 500 or at least 600 or at least 700 or at least 800 or at least 1000 or at least 1200 or at least 1400 or at least 1500 contiguous nucleotides of any sequence of SEQ ID NO: 14-26 (and, optionally, also have a recited percent identity).

The genetic modification can be to a gene or nucleic acid sequence that encodes a WD40 repeat containing protein having a polypeptide sequence of any one of SEQ ID NO: 1-13 or a variant of any of said nucleic acid sequences, and the genetic modification can result in the higher lipid productivity and/or higher biomass productivity as described herein. In various embodiments the genetic modification can be to a nucleic acid sequence that encodes a WD40 repeat containing protein (or domain) having at least 60% sequence identity or at least 70% sequence identity or at least 75% or at least 80% or at least 85% or at least 90% or at least 95% or at least 98% sequence identity to any of SEQ ID NOS: 1-13. In some embodiments the nucleic acid sequence can be a variant as stated and also encode at least 25 or at least 50 or at least 75 or at least 100 or at least 150 or at least 200 or at least 250 or at least 300 or at least 350 or at least 400 contiguous amino acids of any one of SEQ ID NOs: 1-13, or a variant of any.

In any embodiment the WD40 repeat containing protein can be a functional WD40 repeat containing protein. In various embodiments the recombinant algal cell or organism can have a genetic modification described herein to a nucleic acid sequence of SEQ ID NO: 15 or 16, or to a variant of either having at least 70%, or at least 75%, or at least 80%, or at least 85%, or at least 90%, or at least 95%, or at least 98% sequence identity.

Persons of ordinary skill know how to calculate the percent of “sequence identity” between two sequences. Any method of determining sequence identity that has acceptance by most persons of ordinary skill in the art or otherwise widely accepted in the field can be utilized to determine the sequence identity between two sequences. In one embodiment the percent of sequence identity can be determined by BLAST (Basic Local Alignment Search Tool) analysis using the algorithm employed by the programs blastp, blastn, blastx, tblastn, and tblastx (Altschul (1997), Nucleic Acids Res. 25, 3389-3402, and Karlin (1990), Proc. Natl. Acad. Sci. USA 87, 2264-2268). In one embodiment the search parameters for histogram, descriptions, alignments, expect (i.e., the statistical significance threshold for reporting matches against database sequences), cutoff, matrix, and filter (low complexity) can be at the default settings. The default scoring matrix used by blastp, blastx, tblastn, and tblastx can be the BLOSUM62 matrix (Henikoff (1992), Proc. Natl. Acad. Sci. USA 89, 10915-10919). For blastn the scoring matrix can be set by the ratios of M (i.e., the reward score for a pair of matching residues) to N (i.e., the penalty score for mismatching residues), wherein the default values for M and N can be +5 and −4, respectively. Four blastn parameters can be adjusted as follows: Q=10 (gap creation penalty); R=10 (gap extension penalty); wink=1 (generates word hits at every winkth position along the query); and gapw=16 (sets the window width within which gapped alignments are generated). The equivalent Blastp parameter settings for comparison of amino acid sequences can be: Q=9; R=2; wink=1; and gapw=32. A Bestfit comparison between sequences, available in the GCG package version 10.0, can use DNA parameters GAP=50 (gap creation penalty) and LEN=3 (gap extension penalty), and the equivalent settings in protein comparisons can be GAP=8 and LEN=2.

### Laboratory Strains

In various embodiments the one or more genetic modification(s) can be made in (i.e., derived from) a cell or organism that is a wild type, parent, or laboratory strain. Laboratory strains are cells or organisms that have been cultured in a laboratory setting. As a result of culturing over a period of time the strain has undergone some adaptation(s) advantageous to growth in the laboratory setting. A laboratory strain therefore can develop one or more advantageous characteristics from adaptations deriving from time of culturing in the laboratory environment. For example, laboratory strains can have higher biomass productivity and/or higher lipid productivity than a wild-type strain. In some embodiments one or more genetic modifications disclosed herein can be performed on a laboratory strain to result in a recombinant algal cell or organism of the invention. In such embodiments the laboratory strain can therefore be a corresponding control algal cell or organism described herein that does not have the genetic modification being considered. A test cell can be derived from the corresponding control cell or organism. For example, the corresponding control cell or organism can be a parental strain of the test cell or organism.

### Increased Lipid Productivity

The recombinant mutant algae of the invention having a genetic modification to a gene or nucleic acid sequence encoding a WD40 repeat containing protein or other modification described herein can demonstrate an increase in the production of lipid in the cell or organism versus a corresponding (control) cell or organism. The increase in lipid production can be measured by any accepted and suitable method, for example using fatty acid methyl ester (FAME) analysis. In one embodiment the increase in lipid production is measured as an increase in total FAME produced by the recombinant organisms. The recombinant cells or organisms of the invention have a genetic modification to a gene or nucleic acid sequence encoding a WD repeat containing protein or domain and can exhibit at least 20% or at least 25% or at least 30% or at least 40% or at least 50% or at least 60% or at least 70% or at least 80% or at least 90% or at least 100% greater lipid productivity compared to a corresponding control cell or organism, as described herein. In other embodiments the increase in lipid productivity can be 15-25% or 15-35% or 15-45% or 15-50% or 20-25%, or 25-45%, or 25-55%, or 25-70%, or 25-90%, or 25-100%, or 25-150% or 25-200% or 30-35% or 30-45% or 30-55%. The increase can be weight for weight (w/w) of lipid. In any embodiment the lipid can be fatty acid. In one embodiment lipid productivity is measured using the FAME profile (fatty acid methyl ester) of the respective cells or organisms. In one embodiment lipid productivity can be expressed as mg/L. In other embodiments the recombinant cells or organisms of the invention can exhibit at least 50 g/m2 or at least 60 or at least 70 or at least 80 grams per square meter of FAME accumulation after 5 days of cultivation. Methods of producing a FAME profile are known to persons of ordinary skill in the art. A FAME profile can be determined using any suitable and accepted method, for example a method accepted by most persons of ordinary skill in the art. The recombinant cell or organisms of the invention can, optionally, also have an increase in biomass productivity can be 15-35% or 15-40% or 25-45% or 15-50% or 25-70% or 50-100% or 50-200% (w/w).

An increase in lipid production or lipid productivity can be measured by weight, but can also be measured in grams per square meter per day of the surface of a cultivation vessel (e.g., a flask, photobioreactor, cultivation pond). In various embodiments the recombinant cell or alga of the invention produces at least 3 or at least 4 or at least 5 or at least 6 or at least 7 or at least 8 or at least 10 or at least 12 or at least 13 or at least 14 grams per square meter per day of lipid production, which can be measured by the FAME profile. In any of the embodiments the high lipid and/or high biomass productivity phenotype can be obtained under nitrogen deplete conditions, which in some embodiments can involve dilution and/or replacement of medium with fresh nitrogen deplete medium during growth. Dilutions can be by any suitable amount, for example dilution by about 50% or by about 60% or by about 70% or at least 70%, or by about 80%, or by more than 80%, which can be done every day, every 2 days, every 3 days or every 4 days or every 5 days. In one embodiment the lipid product is a fatty acid and/or a derivative of a fatty acid. In one embodiment the fatty acids and/or derivatives of fatty acid comprise one or more species of molecules having a carbon chain between C8-C12, and/or C8-C18 and/or C8-C20 and/or C8-C22 and/or C8-C24, hereby disclosed in all possible combinations and sub-combinations. In one embodiment the growth conditions can be batch growth, involving spinning cells to remove nitrogen from the medium, replacing with nitrogen deplete medium, and resuming batch growth.

In any of the embodiments the genetic modification to the gene or nucleic acid sequence encoding a WD repeat containing protein can result in an attenuation of expression of the respective genes. The genetic modification of any one or more of these genes or nucleic acids can be any of those described herein, e.g., any one or more of SEQ ID NOs: 14-26. In one embodiment the genetic modification is a deletion, disruption, or inactivation. In another embodiment the genetic modification is a deletion (which optionally, can be a functional deletion) or a disruption of the gene.

### Biomass Productivity

The recombinant algal cells of the invention having a genetic modification to a gene or nucleic acid sequence encoding a WD40 repeat containing protein described herein, and can also have higher biomass productivity than a corresponding (control) organism not having the genetic modification. Biomass can be measured as an increase in the total organic carbon (TOC) produced by the cell or organism, for example measured by TOC analysis known to persons of ordinary skill in the art. The recombinant cells can have at least 12% higher, or at least 15% higher, or at least 20% higher or at least 25% higher or at least 30% higher or at least 35% higher, or at least 50% higher or at least 60% higher or at least 70% higher or at least 80% higher or at least 90% higher or at least 100% higher or at least 125% higher or at least 150% higher biomass productivity than a corresponding (control) cell or organism, which can be measured by total organic carbon analysis. In other embodiments the biomass productivity can be 10-15% higher or 15-25% higher or 20-25% higher or 20-30% higher or 20-40% higher or 20-45% higher. The increase in biomass productivity can, optionally, be in addition to the increase in lipid productivity.

Various methods of measuring total organic carbon are known to persons of ordinary skill in the art. Biomass productivity can be measured as mg/ml of culture per time period (e.g., 1 day or 2 days or 3 days or 4 days or 5 days). In some embodiments the higher biomass productivity and/or higher lipid productivity as described herein can occur under nitrogen deplete conditions. Thus, in one embodiment the recombinant alga or cell of the invention can have higher lipid production and/or higher total organic carbon production than a corresponding (control) cell or organism, which higher amount can be produced under nitrogen deplete or low nitrogen conditions. Nitrogen deplete conditions can involve culturing in a buffer having less than 0.5 mM of nitrogen in any available form external to the cell or organism. In some embodiments the cells can be cultured in 0.5 mM or less of KNO3 or urea as a nitrogen source. Other buffers may also be used and be nitrogen deplete [[if they contain a level of nitrogen that does not change the physiology of a nitrogen-related parameter (e.g., lipid productivity or biomass productivity) by more than 10% versus culturing the cell in a medium free of a nitrogen source external to the cells or organisms.]] In any embodiment biomass productivity can be evaluated by measuring an increase in the total organic carbon of the cells. Nutrient replete conditions are those where the growth of the cultivated organism is not limited by a lack of any nutrient. A nitrogen free medium is also considered nitrogen deplete conditions.

### Methods of Producing Lipid

The invention also provides methods for producing a lipid containing product. The methods involve culturing a recombinant algal cell or organism described herein to thereby produce a lipid product. Any of the methods can also involve a step of harvesting lipid produced by the recombinant algal cell or organism described herein. The culturing can be for a suitable period of time, for example, at least 1 day or at least 3 days or at least 5 days.

The invention also provides methods for producing a composition containing lipids. In one embodiment the methods can involve performing a genetic modification to an algal organism in a gene or nucleic acid sequence encoding a WD40 repeat containing protein. The methods can also involve a step of culturing a recombinant algal cell or organism described herein to thereby produce a composition containing lipids. The composition can be a biomass composition or biomass product containing any one or more recombinant algae described herein. The cultivating can be done in any suitable medium conducive to algal growth (e.g., an algal growth medium or any medium described herein). The methods can also involve a step of harvesting lipids from the composition or biomass containing lipids. The methods can involve a step of harvesting lipids from the recombinant cells or organisms. Any of the methods herein can also involve a step of purifying the lipid containing composition to produce a biofuel or biofuel precursor. A biofuel precursor is a composition containing lipid molecules that can be purified or otherwise converted into a biofuel.

The invention also provides methods of producing a recombinant algal cell or organism having higher lipid productivity and/or higher biomass productivity than a corresponding control cell or organism. The methods involve exposing algal cells or organisms to ultraviolet light to produce a recombinant cell or organism described herein that has higher lipid productivity than a corresponding control cell or organism. In one embodiment algal organisms having higher lipid productivity can be identified by contacting the recombinant cells with a stain that identifies lipids (e.g., by BODIPY dye). Optionally methods can include a step of isolating lipids from the recombinant algal organisms. Organisms having higher biomass productivity can be identified by TOC analysis. The recombinant alga can be cultivated in any suitable growth media for algae, such as any of those described herein. The uv treatment can involve, for example, subjecting the culture to uv light, or gamma radiation, or both, for a suitable period of time or under a suitable uv regimen or gamma radiation regimen. Persons of ordinary skill understand suitable regimens for uv exposure for mutagenesis. The uv regimen can involve exposing the cells or organisms to uv radiation, which can be performed in batches with each batch receiving a dose. Multiple cell batches can receive different doses of energy for each batch of cells. Subjecting the algal organism to ultra-violet light can comprise exposing the cell or organism to at least 10 or at least 15 uJ/cm2 of energy, which exposure can be performed within 1 second or within 5 seconds or within 30 seconds or within 1 minute. In some embodiments 4 or 5 batches of cells can receive doses of exposure to 16-57 uJ/cm2 of energy, and exposure energy can increase with each separate batch. The cell batches can be pooled together after exposures are complete. The recombinant alga (or pooled algae) can be cultivated for at least 2 days or at least 3 days, or at least 4 days, or at least 5 days, or at least 6 days, or at least 10 days, or at least 20 days, or from 2-10 days, or from 2-20 days or from 2-25 days after exposure. The recombinant algal organisms can be any described herein. The uv regimen can involve exposing the cells or organisms to multiple exposures to uv radiation, which can become progressively stronger.

Any of the recombinant cells or organisms of the invention can be cultivated in batch, semi-continuous, or continuous culture to produce the higher biomass productivity and/or higher lipid productivity. In some embodiments the culture medium can be nutrient replete, or nitrogen deplete (—N). In some embodiment the culturing is under photoautotrophic conditions, and under these conditions inorganic carbon (e.g., carbon dioxide or carbonate) can be the sole or substantially the sole carbon source in the culture medium.

The invention also provides a biofuel comprising a lipid product of any of the recombinant cells or organisms described herein. The biofuel is produced by purifying a lipid containing composition produced by a recombinant algal cell or organism described herein. The biofuel can be produced by purifying or otherwise converting a lipid product produced by a recombinant algal cell or organism described herein to produce a biofuel. In one embodiment the biofuel is a biodiesel fuel, which can contain fatty acid methyl ester molecules in sufficient proportion to be combustible in an internal combustion engine. In various embodiments the biofuel can contain carbon chains with an average carbon chain length of at least 5 carbon atoms, or at least 8 carbon atoms, or at least 10 carbon atoms.

### FAME and TOC Analysis Methods

The lipid productivity of the cells or organisms can be measured by any method accepted in the art, for example as an increase or decrease in fatty acid methyl esters comprised in the cell, i.e., FAME analysis. In various embodiments any of the recombinant algal cells or organisms of the invention can have higher biomass productivity as described herein versus corresponding control cells or organisms. In some embodiments the recombinant algal cells or organisms of the invention can have higher lipid productivity and also higher biomass productivity compared to a corresponding control cell or organism. Biomass productivity can be measured by any methods accepted in the art, for example by measuring the total organic carbon (TOC) content of a cell. Embodiments of both methods are provided in the Examples.

“FAME lipids” or “FAME” refers to lipids having acyl moieties that can be derivatized to fatty acid methyl esters, such as, for example, monoacylglycerides, diacylglycerides, triacylglycerides, wax esters, and membrane lipids such as phospholipids, galactolipids, etc. In some embodiments lipid productivity is assessed as FAME productivity in milligrams per liter (mg/L), and for algae, may be reported as grams per square meter per day (g/m2/day). In semi-continuous assays, mg/L values are converted to g/m2/day by taking into account the area of incident irradiance (the SCPA flask rack aperture of 1½ inches×3⅜″, or 0.003145 m2) and the volume of the culture (550 ml). To obtain productivity values in g/m2/day, mg/L values are multiplied by the daily dilution rate (30%) and a conversion factor of 0.175. Where lipid or subcategories thereof (for example, TAG or FAME) are referred to as a percentage, the percentage is a weight percent unless indicated otherwise. The term “fatty acid product” includes free fatty acids, mono-di, or tri-glycerides, fatty aldehydes, fatty alcohols, fatty acid esters (including, but not limited to, wax esters); and hydrocarbons, including, but not limited to, alkanes and alkenes).

### Embodiments

In one embodiment the invention provides a recombinant algal organism of the Class Trebouxiophyceae having a genetic modification in a gene or nucleic acid sequence encoding a WD40 repeat containing protein. The recombinant alga exhibits higher lipid productivity and/or biomass productivity versus a corresponding control algal cell not having the genetic modification. In various embodiment the Trebouxiophyceae organism can be from the family Oocystaceae or Chlorellaceae. In one embodiment the organism is of the genus Oocystis.

In one embodiment the invention provides a recombinant Trebouxiophyceae organism having a deletion, disruption, or inactivation in a gene or nucleic acid sequence encoding a WD40 repeat containing protein. The deletion can be a functional deletion. In one embodiment the deletion, disruption, or inactivation involves the insertion of a nonsense mutation or a frameshift mutation in a gene or nucleic acid sequence encoding a WD40 repeat containing protein. In one embodiment the encoded WD40 repeat containing protein can have at least 80% or at least 85% or at least 90% or at least 95% sequence identity to SEQ ID NO: 1 or 8. In another embodiment the recombinant cell or organism can have the deletion, disruption, or inactivation in a nucleic acid sequence encoding a WD40 repeat containing protein, which be a nucleic acid having at least 80% or at least 90% or at least 95% sequence identity to SEQ ID NO: 15 or SEQ ID NO: 16. The recombinant alga exhibits higher lipid productivity and/or biomass productivity versus a corresponding control algal cell not having the genetic modification. The alga can be a Trebouxiophyceae organism, for example from the family Oocystaceae, and optionally from the genus Oocystis. The increase in lipid productivity can be an increase in 2-day FAME accumulation of at least 20% w/w, or at least 25%, or 30-50% or 30-55%, which can, optionally, be cultivated under nitrogen deplete conditions. The recombinant cells or organisms can, optionally, also have an increase in biomass productivity of at least 12% or at least 15% or at least 20% or 15-25% or 20-40%, which can be expressed as 2-day TOC accumulation. Thus in one embodiment the recombinant cells or organisms have an increase in lipid productivity (as measured by FAME profile) of 30-55% and an increase in biomass productivity of at least 20% versus a control cell or organism. In another embodiment the increase in lipid productivity can be 20-40%. The increase in FAME accumulation and/or TOC accumulation can be measured after two days or after five days cultivation, and can be in nitrogen deplete conditions. The recombinant cells or organisms can also have a FAME/TOC ratio of greater than 0.3 after 2 days of cultivation.

In another embodiment the cells or organisms of the invention can have an increase in lipid productivity of at least 20% (as measured by 2-day FAME accumulation) and, optionally, an increase in biomass productivity of at least 12% (as measured by TOC); and optionally also have a FAME/TOC ratio of greater than 0.3 or 0.4. In another embodiment the cells or organisms of the invention can have an increase in lipid productivity of at least 20% (as measured by 2-day FAME accumulation); and optionally also have a FAME/TOC ratio of greater than 0.5.

In one embodiment the invention provides a recombinant algal organism of the Class Trebouxiophyceae having a genetic modification to a gene or nucleic acid sequence encoding a WD40 repeat containing protein. In one embodiment the gene or nucleic acid sequence is any one of SEQ ID NO: 14-26 or a variant of any, or can be a gene or nucleic acid sequence encoding any one of SEQ ID NO: 1-13, or a variant of any. The genetic modification can be a deletion (optionally a functional deletion) or disruption of the gene or nucleic acid sequence. The deletion can be a functional deletion. The recombinant alga exhibits higher lipid productivity and, optionally, higher biomass productivity versus a corresponding control algal cell not having the genetic modification. In various embodiment the Trebouxiophyceae organism can be from the family Oocystaceae or Chlorellaceae. In one embodiment the organism is of the genus Oocystis. The increase in lipid productivity can be an increase in 2-day or 5-day FAME accumulation of at least 20% w/w, or 30-50% or 30-55%, which can, optionally, be cultivated under nitrogen deplete conditions. The recombinant cells or organisms can, optionally, also have an increase in biomass productivity of at least 15% or at least 20% or 15-25% or 20-40%, which can be expressed as 2-day or 5-day TOC accumulation. Thus in one embodiment the recombinant cells or organisms have an increase in lipid productivity of 30-55% and an increase in biomass productivity of at least 20%. In another embodiment the increase in lipid productivity can be 20-40%. The increase in FAME accumulation and/or TOC accumulation can be measured after two days or after five days cultivation in nitrogen deplete conditions. In another embodiment the cells or organisms of the invention can have an increase in lipid productivity of at least 50% (as measured by 5-day FAME accumulation); and optionally also have a FAME/TOC ratio of greater than 0.5 or 0.6 after 5 days cultivation.

In all embodiments the increase in lipid productivity and/or TOC productivity can be relative to a corresponding (control) cell or organism, which can be a wild-type cell or a laboratory strain.

### Example 1

Various aliquots of 10 mL of Strain 15 (STR00015 or “wild type”) Oocystis sp. were acclimated to diel growth conditions on urea supplemented minimal media. Different aliquots of cells at a concentration of 2×10⁶ cells/mL were then exposed to different amounts of UV radiation (22.4, 33.6, 44.8 and 56 mJ/cm² using at total of 50, 50, 50 and 20 mL of culture, respectively) in a UV crosslinker apparatus. The mutagenized cell aliquots were allowed to recover in the dark for 48 hrs. Cultures were then scaled up in low light (˜100 uE) before a first round of enrichment. The mutagenized cells were then acclimated to diel growth at a light intensity of ˜100 uE and 1% CO₂ in urea supplemented minimal medium for a week. The cultures were scaled up for 3 days, bubbled with 1% CO₂ at a maximum irradiance of ˜1400 uE under diel conditions, to an OD730 of about 1.0. The cultures was then centrifuged at 5000 g for 10 mins and the cell pellets resuspended in nitrogen-free minimal medium to an OD730 of about 0.9. This nitrogen-free culture was then incubated for 48 hrs in square-bottom flasks bubbled with 1% CO₂ at a maximum irradiance of about 1400 uE under diel conditions. After 48 hours in nitrogen-free batch growth, an aliquot of cells was removed and subjected to staining with the lipid specific dye BODIPY for 10 mins in the dark at a final concentration of 0.2 ug/mL.

Mutant cells having the highest level of BODIPY staining were enriched by fluorescence activated cell sorting (FACS). Enriched cell populations were scaled up and starved for nitrogen as described above, and then subjected to additional rounds of BODIPY-based FACS enrichment. This iterative process was repeated for a total of five rounds, with the top 2%, 1%, 1%, 0.25% and 0.25% of the population retained in each iteration.

The final sort was plated on minimal medium agar plates supplemented with urea to isolate single axenic colonies.

Isolates were scaled up in T25 tissue culture flasks in minimal medium supplemented with urea, then transitioned to nitrogen-free minimal medium for 48 hrs. The lipid and biomass accumulation of isolated mutants were compared to the parental strain STR00015, with lipid content measured by total fatty acid methyl ester (FAME) analysis and biomass measured by total organic carbon (TOC). As can be seen in FIG. 1, several isolates from the screen showed an increase in accumulated FAME and TOC, as well as FAME/TOC—an indicator of how much fixed carbon is partitioned to lipids. This indicated that mutants with improved lipid productivity had been isolated by the process described. Strain 27434 was identified as having high FAME and biomass accumulation.

### Example 2

Genomic DNA was isolated from Strain 27434 along with parental (wild type) Strain 15 and sequenced on high throughput sequencing system generating 150 bp paired end reads. Reads were processed, mapped to the wild type strain as reference genome and analyzed by a small variants algorithm. An example of a small variants algorithm is the Freebayes polymorphism detection software, although other programs can also be successfully utilized. Analysis of SNPs and small insertions/deletions (InDels) revealed Strain '434 contained a total of 170 polymorphisms. Twenty-four of these mutations were located within exons or at splice junctions and were prioritized for Cas9-mediated gene deletion as they had the highest probability of altering gene function and/or activity, as shown in Table 1 below.

TABLE 1
Mutations identified within exons or at splice junctions in Strain ′434

| | Transcripts | Descriptions | Type | Ref | Alt | AAMod |
|---|---|---|---|---|---|---|
| 1 | 3EUKT2015873 | Protein kinase-like (PK-like) | SNP | G | A | Gln1301* |
| 2 | 3EUKT2027500 | YVTN repeat-like/Quinoprotein amine | Deletion | AGACTC | AG | Val204fs |
| | | dehydrogenase | | GCACCG | | |
| 3 | 3EUKT2031181 | CAAX amino terminal protease self- | SNP | G | A | Gln48* |
| | | immunity | | | | |
| 4 | 3EUKT2028953 | Proteasome subunit alpha type-1-A | SNP | C | T | Leu546Phe |
| 5 | 3EUKT2014620 | 3-methylcrotonyl-CoA carboxylase alpha | SNP | C | A | Ala426Ser |
| | | subunit | | | | |
| 6 | 3EUKT2012690 | DNA-directed DNA polymerase family | SNP | G | A | Ala627Val |
| | | protein | | | | |
| 7 | 3EUKT2036647 | Conserved predicted protein | SNP | C | T | Glu640Lys |
| 8 | 3EUKT2016841 | Conserved predicted protein | SNP | T | A | Leu332Gln |
| 9 | 3EUKT2025932 | Chitin binding Peritrophin-A domain | SNP | C | T | Pro239Leu |
| 10 | 3EUKT3238737 | Retrovirus-related Pol polyprotein from | SNP | G | A | Ser895Phe |
| | | transposon 17.6 | | | | |
| 11 | 3EUKT2017815 | Phosphoglycerate mutase-like protein | Complex | CC | TT | Gly235Glu |
| 12 | 3EUKT2020372 | Conserved predicted protein | SNP | A | G | Thr118Ala |
| 13 | 3EUKT2011248 | RAP domain | SNP | G | A | Gly1722Asp |
| 14 | 3EUKT2015114 | Conserved predicted protein | SNP | G | A | Arg576His |
| 15 | 3EUKT2013326 | Manganese-dependent ADP-ribose/CDP- | SNP | G | A | Ser174Phe |
| | | alcohol diphosphatase | | | | |
| 16 | 3EUKT2015233 | Phosphatidylinositol (PI) | SNP | C | T | Ser106Phe |
| | | phosphodiesterase | | | | |
| 17 | 3EUKT2015577 | T-complex 1 subunit beta | SNP | G | A | Glu265Lys |
| 18 | 3EUKT2015261 | Cell surface protein homologous to | SNP | C | T | Pro171Ser |
| | | bacterial outer membrane proteins | | | | |
| 19 | 3EUKT2026772 | Conserved predicted protein | SNP | G | A | Pro18Ser |
| 20 | 3EUKT2021194 | Conserved predicted protein | SNP | C | T | Pro48Leu |
| 21 | 3EUKT2021524 | Conserved predicted protein | SNP | C | T | Ser19Leu |
| 22 | 3EUKT2018868 | Conserved predicted protein | SNP | T | A | Asn84Ile |
| 23 | 3EUKT2022105 | Axonemal inner arm dynein heavy chain | SNP | G | A | Ala202Val |
| | | 4 | | | | |
| 24 | 3EUKT2018529 | Ribonuclease Inhibitor | SNP | G | A | Splice |
| | | | | | | junction |

The remaining 146 mutations were either intergenic or present in introns of a gene. An assessment of transcriptomics data from Strain 15 (wt) and Strain 27434 indicated that none of these 146 mutations had any significant impact on gene expression or transcript splicing.

### Example 3

### Identification of Mutations

To identify which mutation(s) caused the high lipid phenotype in Strain 27434, independent knockouts of genes bearing SNPs in the strain (Table 1) were conducted via RNP-based Cas9-mediated gene disruption in two background strains: (i) the Strain 15 wild type parental strain; and (ii) Strain 24194—a laboratory strain evolved from Strain 15 and having improved biomass and lipid productivity.

All the strains generated were tested for improved biomass and lipid accumulation during nitrogen starvation in T25 flasks. From this analysis three independent lines were identified having a deletion of 3EUKT2027500, a gene encoding a WD40 repeat containing protein. These lines showed significantly higher biomass and lipid accumulation compared to the parental lines. One of these was constructed in the Strain 15 (wt) control strain (FIG. 2), while the other two were constructed in the Strain 24194 control strain (FIG. 3). As shown in FIG. 2, the first genetically engineered strain (identified as Strain 29857) showed a 22% and 15% improvement in accumulated FAME and TOC, respectively, compared to Strain 15. The strains engineered in the Strain 24194 control (identified as Strains 30525 and 30526) showed a 22% increase in FAME accumulation (FIG. 3).

### Example 4

Large scale productivity testing in 500 mL square-bottom flasks was conducted as described above, but over a 5 day period. The study revealed that the two knockout lines in laboratory Strain 24194 (Strain 30525 and 30526) showed a 14% and 21% improvement in lipid productivity, respectively (FIG. 4), confirming the results in the T25 flasks. The measured FAME/TOC was also substantially higher on each day of the experiment for both engineered strains (FIG. 4). The data therefore showed that deletion of 3EUKT2027500 provides a substantial increase in carbon partitioning and lipid productivity.

### Example 5

The amino acid sequence of 3EUKT2027500 (#2 in Table 1) was analyzed for functional domains and orthologs in other species. 3EUKT2027500 encodes a WD40 repeat containing protein with four tandem domains with weak similarity to WD40 repeats. WD40 repeats are short minimally conserved structural motifs of about 40 amino acids often beginning with a glycine-histidine (GH) dipeptide and ending with tryptophan-aspartic acid (WD) dipeptide. Multiple copies of these repeats fold together to form the WD40 domain, which functions as a scaffold enabling interaction with proteins and nucleic acid and facilitating the formation of multiprotein complexes. WD40 repeat containing proteins are involved in a variety of cellular processes, including cell cycle progression, transcriptional regulation, signal transduction, apoptosis, biosynthesis of plant cell walls, anthocyanin biosynthesis, etc.

Further sequence identity analysis summarized in Table 2 revealed orthologs of 3EUKT2027500 are highly conserved and broadly distributed in green algae and plants.

TABLE 2
Alignment of Entire Protein Sequences
of 3EUKT2027500 Orthologs

| | | | | % |
|---|---|---|---|---|
| | | | % | Similarity |
| | Organisms | Gene/Seq ID | Identity | [Positives] |
| 1 | Coccomyxa | XP_005642574.1 | 78 | 89 |
| | subellipsoidea | SEQ ID NO: 2 | | |
| 2 | Chlamydomonas | Cre16.g674000 | 69 | 78 |
| | reinhardtii | SEQ ID NO: 3 | | |
| 3 | Volvox carteri | XP_002950453.1 | 72 | 80 |
| | | SEQ ID NO: 4 | | |
| 4 | Auxenochlorella | RMZ56784.1 | 62 | 76 |
| | protothecoides | SEQ ID NO: 5 | | |
| 5 | Chlorella | PRW57951.1 | 70 | 81 |
| | sorokiniana | SEQ ID NO: 6 | | |
| 6 | Chlorella variabilis | XP_005847572.1 | 69 | 80 |
| | | SEQ ID NO: 7 | | |
| 7 | Parachlorella | 3EUKT595038 | 73 | 85 |
| | sp WT1185 | SEQ ID NO: 8 | | |
| 8 | Picochlorum celeri | 3EUKT2133049 | 44 | 62 |
| | | SEQ ID NO: 9 | | |
| 9 | Tetraselmis | 3EUKT668672 | 73 | 82 |
| | | SEQ ID NO: 10 | | |
| 10 | Ostreococcus | XP_001417471.1 | 71 | 86 |
| | lucimarinus | SEQ ID NO: 11 | | |
| 11 | Micromonas commoda | XP_002507884.1 | 72 | 87 |
| | | SEQ ID NO: 12 | | |
| 12 | Arabidopsis | AT1G12910 | 70 | 83 |
| | | SEQ ID NO: 13 | | |

An alignment study of the protein sequences from the Table 2 orthologs was conducted and showed a high degree of sequence conservation across Chlorophyte algae species and across the entire length of the proteins, and particularly in key domains as illustrated in FIG. 5. Sequence similarity was calculated using the bioinformatics matrix BLOSUM62 for sequence alignment.

It will be readily apparent to one skilled in the art that varying substitutions and modifications may be made to the invention disclosed herein without departing from the scope and spirit of the invention.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described. Other embodiments are within the following claims.

What is claimed is:

1. A method of producing a composition containing lipids comprising,
performing a deletion, disruption, or inactivation in an algal organism in a gene encoding a WD40 repeat containing protein having at least 90% sequence identity to the polypeptide of SEQ ID NO: 1 to product a recombinant algal organism;
culturing the organism, and thereby producing a composition containing lipids.

2. The method of claim 1 further comprising harvesting the composition containing lipids from the recombinant algal organism.

3. The method of claim 1 wherein the gene encoding the WD repeat containing protein comprises a nucleic acid sequence having at least 90% sequence identity to SEQ ID NO: 16.

4. The method of claim 1 wherein the recombinant algal organism has at least 30% greater lipid productivity versus a control alga that does not have the deletion, disruption, or inactivation.

5. The method of claim 1 wherein the recombinant alga exhibits at least 40 grams per square meter per day of lipid production after 5 days of cultivation.

6. The method of claim 1 wherein the deletion, disruption, or inactivation is a frame shift mutation.

7. The method of claim 1 wherein the deletion, disruption, or inactivation is a knock out mutation.

8. The method of claim 1 wherein the deletion, disruption, or inactivation is performed by mutagenesis.

9. The method of claim 1 wherein the culturing is performed under nitrogen deficient conditions.

10. The method of claim 1 wherein the recombinant alga has higher biomass productivity per unit time as measured by production of total organic carbon (TOC) versus a control alga that does not comprise the deletion, disruption, or inactivation.

11. The method of claim 10 wherein the recombinant alga has at least 15% higher biomass productivity.

12. The method of claim 1 wherein the algal organism is a Chlorophyte alga.

13. The method of claim 12 wherein the Chlorophyte alga is of the class Trebouxiophyte.

14. The method of claim 13 wherein the recombinant algal organism is an alga of the genus Oocystis.

15. The method of claim 1 further comprising:
mutagenizing a population of the algal organisms;
screening the mutagenized algal organisms for higher lipid productivity;
sequencing at least a portion of the genome of the mutagenized algal organisms;
identifying a deletion, disruption, or inactivation in the gene encoding a WD40 repeat family protein compared to the population of algal organisms prior to mutagenesis; and
recapitulating the deletion, disruption, or inactivation in a parental strain of the mutagenized algal organisms.

16. The method of claim 15 further comprising harvesting a lipidic composition from the algal organism.

17. The method of claim 15 wherein the algal organism is a Chlorophyte alga.

18. The method of claim 15 wherein the Chlorophyte alga is of the class Trebouxiophyte.

19. The method of claim 18 wherein the recombinant algal organism is an alga of the genus Oocystis.


SEQUENCE LISTING (26 sequences)

SEQ ID NO: 1 - protein, 340 aa, Oocystis sp.
  REGION 1..340: note = misc_feature - WD40 repeat protein, 3EUKT2027500
MAEPDANGASDGKRAEIYTYEFPNLVYSMNWTSRRDKKFRLAVGSFIEDYNNVVNIISLDEEQGKFVCDPSLTFKHPYPPTKVMFVPDREGTRPDLLATTGDYLRVWKIGEDGVTLQKLLNDNKNSEFCAPLTSFDWNETDPKRLGTSSIDTTCTIWDIEKGVVDTQLIAHDKEVYDIAWGGVGVFASVSADGSVRVFDLRDKEHSTIIYETPSPETPLLRLGWNKQDPRYMATIVMDSNRVVVLDIRVPTVPVAELQRHQACANALAWAPHSSCHICTAGDDAQALIWDLSAVSKEGDSGLDPILAYNAGQEVNQLQWSSTQPDWVAVCFGNKAQILRV

SEQ ID NO: 2 - protein, 335 aa, Coccomyxa sp.
  REGION 1..335: note = misc_feature - WD40 repeat protein
  source 1..335: note = Coccomyxa subellipsoidea
MDGRLNDRRAEIYTYDSENIVYGLSWSNRRDKKFRLAVGSFIEEYDNYVEIITLDDATCKFTSDAQLAFQHPYPPTKIMFMPDKEGAQPDLLATTGDYLRIWQLKEDGTQLVKLLNNNKNSEFCAPLTSFDWNETDLNRLGTSSIDTTCTIWDIEKGVVDTQLIAHDKEVYDIAWGGVGVFASVSADGSVRVFDLRDKEHSTIIYDSPQPDTPLLRLGWNKQDPRYMATVLMDSTKVVILDIRYPTLPVAELQRHQAPVNAVAWAPHSSCHICSAGDDAQALIWDLSSMSRPMDQTLDPILAYSAGAEVNQLQWSTTQPDWVAICFANKTQILRV

SEQ ID NO: 3 - protein, 359 aa, Chlamydomonas reinhardtii
  REGION 1..359: note = misc_feature - WD40 repeat protein
MSASDKRERQEVYTYVAPDPVYAMNWSVRRDKRFRLGVASFREDVTNYVDIVSLDDESDELRADPGLRFPHDYPATKLMWMPDREGCRPDLLATTGEALRIWRVCDGSEGEESGSGPGGRGVQLRSLLNNNKQSEFSAPLTSFDWNEADPKRLGTSSIDTTCTIWDIEKGEVDTQLIAHDREVYDIAWGGLGVFATVSADGSVRVFDLRDKEHSTIIYESPQPDTPLLRLGWNRQDPRYMATILQDSPKVVILDIRYPTLPVAELCRHQAPVNALAWAPHSAQHICTAGDDSQALIWDVSAVGGGNNANAAAGGGASDVSLDPILAYGAASEVNQLQWSSAQPDWVAICFGNKTQILRV

SEQ ID NO: 4 - protein, 351 aa, Volvox sp.
  REGION 1..351: note = misc_feature - WD40 repeat protein
  source 1..351: note = Volvox carteri
MSNSDKRAEIYTYVAQDPVYAMNWSVRRDRRFRLAVGSFREDVTNYVEIISLDDDAAELRSDPSLRFHHDYPATKLMWLPDREGCRPDLLATTGEALRIWRVLDPDSVAGDGEDLRALLNNNKQSEFSAPLTSFDWNEADPKRLGTSSIDTTCTIWDIEKGEVDTQLIAHDREVYDIAWGGLGVFATVSADGSVRVFDLRDKEHSTIIYESPQPDTPLLRLGWNRQDPRYMATILMDSPKVVILDIRYPTLPVAELHRHQAPVNALAWAPHSAQHICTAGDDSQALIWDVSAVGSGGGQPGALGGGTAGDVSLDPILAYGAQSEVNQLQWSSAQPDWVAICFANKTQILRV

SEQ ID NO: 5 - protein, 341 aa, Chlorella protothecoides
  REGION 1..341: note = misc_feature - WD40 repeat protein
  source 1..341: note = Auxenochlorella protothecoides
MSGPSGDKRAEIYTHESADPIYALNWSVRTDKPFRLATGSYVEDQNNHIDIIVLDEAREQFQADPRLSFVHPFPATKLMFLPVKDPNQPDLLATTSDFLRIWSISEDGVALEKLLNNTKTSEYCEPITSFDWNHLEPRRLGTASLDATCTVWDIERGCVDTQLIAHDGEVYDLAWGGATMFASVSADASVRVFDLRDRDHSTITYESRGGEALVRLGWNRADPRFMAVLAAGSAEVVVLDVRRPAAPLARLARHTAPANVLAWAPHSACHLCSAGDDGAALIWDVGALGGGGGPGGAAQDPGLDPILAYNAGAEVAALQWSAAQPDWVAIAFGNNAQVLRV

SEQ ID NO: 6 - protein, 351 aa, Chlorella sorokiniana
  REGION 1..351: note = misc_feature - WD40 repeat protein
MQQQGEGRAEIYTYESPHLVYGAGWSVRPDKPFRLALGSFIEDYANRVEIVQLDEGRGVIRSNPALGFQHPYPPTKVGFIPDKDGTRPDLLATSGDFLRLWRIHDEPGSNQHVRLEKLLNNNKGGEFSAPLTSFDWNELDPRRIGTASIDTTCTVWDVERGVVDTQLIAHDKEVYDIAWGGVGIFASVSADGSVRVFDLRDKEHSTIIYESPQPSTPLLRLSWNKQDPRYIAAFAMDSSKVLVLDIRYPTLPVAQLQRHQASVNAVCWAPHSAVHLCSAGDDCQALIWDLALSGAMGGQQQDGTAAAAAAGGLDPILAYNAGTEINQLQWSASQPDWVAICFGNKAQILRV

SEQ ID NO: 7 - protein, 355 aa, Chlorella sp.
  REGION 1..355: note = misc_feature - WD40 repeat protein
  source 1..355: note = Chlorella variabilis
MQDQQQQGEGRAEIYTYSSSASVYACGFSSRPDKPFRLAVGSFIDDYANKVEIIQLDEAAGVVRNNPALTFQHPYPPTKVAFIPDKSGTRPDLLATSGDFLRLWRVSDEPGAQQGVRLEKLLNNNKGGDFAAPLTSFDWNELDPRRVGTASIDTTCTVWDVERGVVDTQLIAHDKEVYDIAWGGVGIFASVSADGSVRVFDLRDKEHSTIIYESPQPDTPLLRLSWNKQDPRYIAVLAMDSPRVTVLDIRYPTLPVAELQRHQAGVNAICWAPHSATHLCSAGDDSQALIWDLGLLGTLGQQPEGGPPGAAAAGGGLDPILAYNAGAEVNQLQWSPAQPDWVAICFGNKTQLLRV

SEQ ID NO: 8 - protein, 334 aa, unidentified
  REGION 1..334: note = misc_feature - WD40 repeat protein
  source 1..334: note = Parachlorella sp.
MQRAEIHTYESPTLVYALNWSVRPDKPFRLAIGSYIEDYNNRVEIVTLGEDGNGMRPSPRHTFQHPYPPTKLQFVPDPDGSRPDLLASSGDFLRLWRITEDGVSLEKLLNNNKASEFCAPLTSFDWNENDPKRVGTASIDTTCTVWDIEKGVVDTQLIAHDKEVYDIAWGGVGVFASVSADGSVRVFDLRDKEHSTIIYESPQPDTPLLRLAWNKQDPRYMATTALNSSAIVVLDIRFPTVPVVELSKHQAACNAVAWAPQSANHICSAGDDCQALIWDLSTLGEGGAGQAGSPPLDPILSYMAGAEVNQLQWSASHPDWVAICFGNKTQILRV

SEQ ID NO: 9 - protein, 337 aa, unidentified
  REGION 1..337: note = misc_feature - WD40 repeat protein
  source 1..337: note = Picochlorum celeri
MEHPPAPNILTYDSSSIVFALDWSSRQDKGVRVAVGSFVEGVSNTVEILRVTPAGLIVDDKETFGIEYPATQVGFIPDRFCNKPDLLATSGDAVRLWKISDAGTTLELVLNDPKNTSKNFSAVTCFDWSEINVKVLAAGSSAGRLLLWDTESGRLQGTMVGHEDEILDCQWAANDVIVSSSGDGSIRMYDLRDKDHCTVLYETPRRTPVPRFCWNKLDPRHLAFSIEKSRLVSVLDVRFPTEPVILLDGHMGNCTALGWSPHREEYLCSVGDDCHALIWDVGKVNSEEDSKPNREAVDASPILAYNAQAEINAMAWNPIDPDWIAICARNRTQVLRI

SEQ ID NO: 10 - protein, 412 aa, Tetraselmis sp.
  REGION 1..412: note = misc_feature - WD40 repeat protein
MPTSDATQAHEHHHTLAATPTQQANNAAPLADRFTLGLLAMASGPEDRGAGAAGAAPHQRGDSNGKAVTDKRGEIYTYEAPYPVYGMNWSVLLQVREDMKFRLAVGSFVEDVENAVELIRLNEETGKFESNPAHKFVHPYPPTKIMFIPDRDCSRPDLLATTGDYLRLWRVEEDGVTLHKLLTNNKNSEFCAPLTSFDWNEADPRQLGTSSIDTTCTIWDIERGVVDTQLIAHDKEVYDIAWGGQGVFASVSADGSVRVFDLRDKDHSTIIYESGMPEIPLLRLGWNKQDPRYMATILMDSSKVVVLDIRYPTMPVAELEAHHKPVNALAWAPQSSSHICTAGDDAQALIWNLAPMGTQGPMGGAAPAVLGADLDPILAYNAGEEINQLQWSSTQSDWVGISFGNKIQILRI

SEQ ID NO: 11 - protein, 332 aa, unidentified
  REGION 1..332: note = misc_feature - WD40 repeat protein
  source 1..332: note = Ostreococcus lucimarinus
MNAEKRAEIYTYEAPWMIYACNWSVRQDKRFRLALGSFVEEYSNKVEIITLDEETGEFPKEAQCSFTHPYPCTKILFIPDKECTKEDLLATTGDYLRIWQVQDDNTVQMKSLLNNNKSSEFCAPLTSFDWNETKLQRVGTSSIDTTCTIWDIERECVDTQLIAHDKEVYDIAWGGPEVFASVSADGSVRVFDLRDKDHSTIIYESQTPDTPLLRLGWNKQDPRYMATICMDSPVIILDIRFPTLPVAELQSHRASVNTLAWAPHSSSHMCTAGDDSQALIWDLSSMNQPPEGGLDPILAYSAGAEINQLQWSASQPDWISIAFRNSLQILRV

SEQ ID NO: 12 - protein, 336 aa, Micromonas sp.
  REGION 1..336: note = misc_feature - WD40 repeat protein
  source 1..336: note = Micromonas commoda
MAAMGSGQSGAEIYTYEAPWLVYAMNWSVRQDKRFRLALGSFVEEYSNKVEIITLDEQRREFPAEPTHRFDHPYPCTKIMFVPDAEGTSEDLLATSGDYLRVWRIGDDGVHLRSLLNNNKNSDFCAPLTSFDWSTTNLARVGTSSLDTTCTIWDLEKETVDSQLIAHDKEVYDIAWGGPEVFASVSADGSVRVFDLRDKDHSTIVYESPTPDTPLLRLGWNKQNPRYMATMEMDSAKVVVLDIRVPALPVAELKKHRAAVNTLAWAPHSSRNICTAGDDAQALIWDLSSVAQPGEDGMDPMLAYNAGAEISQLQWSATQTDWIAIAFGKNLQVLHV

SEQ ID NO: 13 - protein, 346 aa, Arabidopsis sp.
  REGION 1..346: note = misc_feature - WD40 repeat protein
MGTSSDPIQDGSDEQQKRSEIYTYEAPWHIYAMNWSVRRDKKYRLAITSLLEQYPNRVEIVQLDESNGEIRSDPNLSFEHPYPPTKTIFIPDKECQRPDLLATSSDFLRLWRIADDHSRVELKSCLNSNKNSEFCGPLTSFDWNEAEPRRIGTSSTDTTCTIWDIEREAVDTQLIAHDKEVFDIAWGGVGVFASVSADGSVRVFDLRDKEHSTIIYESSEPDTPLVRLGWNKQDPRYMATIIMDSAKVVVLDIRFPALPVVELQRHQASVNAIAWAPHSSCHICTAGDDSQALIWDISSMGQHVEGGLDPILAYTAGAEIEQLQWSSSQPDWVAIAFSTKLQILRV

SEQ ID NO: 14 - DNA, 12 bp, Oocystis sp.
  misc_feature 1..12: note = partial sequence of WD40 repeat protein
  source 1..12: mol_type = unassigned DNA
agactcgcaccg

SEQ ID NO: 15 - DNA, 1643 bp, Oocystis sp.
  misc_feature 1..1643: note = WD40 repeat protein, 3EUKG2027500, encodes SEQ ID NO: 1
  source 1..1643: mol_type = unassigned DNA
atggccgagccggacgcgaacggcgcgtcggatggcaagcgcgcggagatttacacgtacgagttccccaacctcgtttactccatgaactggacggtaagcaagcacagttgtagcgcacacagccttgggtgctgctgcgtccttgctgatggtcatgcttgcgtcggccacccgttttctcacctaccgaaactggctgcgcacccctttcagttatgcttgtaatcaccgcctcattacctttgtgtgcagtctcgtcgggacaagaagtttcgactggcagtgggcagcttcatcgaggactataataacgtcgtcaatatcatatcctgtacgcgccttccccgcactacccagcgtagcggctcagtctgtcgtgcggggttggcacgacagtcgttgggttgaagactatttgaactattgctgattagtgacgcccatgctctctcgtaaagcggggcggatggctcctttcgcgcccactaccatctggccgcgacctgtggctctgacgcctcgctgggtctcgacctgctgaccatactccctcgttactggctgcaacgcagtggacgaggaacagggcaagtttgtatgcgacccgtcactgaccttcaagcatccgtacccaccgaccaaggtgatgtttgtgccagaccgggaaggcactcggcccgacctgttggccaccaccggcgactatctgcgcgtgtggaagatcggggaggatggcgtcacgctgcagaagctgctgaacgatgtaaggcctagattaacgtccagcgctgtggggaaggaccgacggcacgggccggaaagggacatgcatgccgtaccgggggtgatcacgggcgacggcccaccggatcatcatcttcctcgcttccaccaaccctgcgcaacgtccttcgcaacgtcttgaacaatattttgcatctttcacgttcatccatcctcgtcatggcgaaattaacttgcagaacaagaacagcgagttttgcgcgccgctcacatcgttcgactggaacgagaccgaccccaagcgcctgggcaccagctctatcgataccacgtgcacgatctgggacatcgagaagggcgtggtggacacgcagctcatcgcgcacgacaaggaggtgtatgacatcgcgtggggcggcgtcggcgtcttcgcgtcggtgtccgccgacggctcggtgcgagtcttcgacttgcgagacaaggagcatagcacgatcatctacgagacgccgtcgccagagacgccgctgctgcgcctggggtggaacaaacaggaccccaggtacatggcgacgatcgtgatggactctaaccgcgtggttgtgctggacatccgcgtgcccaccgtgcctgtcgccgagctgcagcggcaccaggcgtgcgcaaacgcgcttgcctgggcgccgcacagcagctgccacatctgcacagcgggcgacgacgcacaggcgctgatctgggacctcagcgcggtatccaaggagggcgactcgggcctggaccccatcctcgcgtacaatgcaggtcaagaggtaaaccagctgcagtggtcttcgacgcagcccgattgggtggcagtctgctttggcaacaaggcgcagatcctgcgagtgtga

SEQ ID NO: 16 - DNA, 1023 bp, Oocystis sp.
  misc_feature 1..1023: note = WD40 repeat protein, cds of 3EUKG2027500, encodes SEQ ID NO: 1
  source 1..1023: mol_type = unassigned DNA
atggccgagccggacgcgaacggcgcgtcggatggcaagcgcgcggagatttacacgtacgagttccccaacctcgtttactccatgaactggacgtctcgtcgggacaagaagtttcgactggcagtgggcagcttcatcgaggactataataacgtcgtcaatatcatatccttggacgaggaacagggcaagtttgtatgcgacccgtcactgaccttcaagcatccgtacccaccgaccaaggtgatgtttgtgccagaccgggaaggcactcggcccgacctgttggccaccaccggcgactatctgcgcgtgtggaagatcggggaggatggcgtcacgctgcagaagctgctgaacgataacaagaacagcgagttttgcgcgccgctcacatcgttcgactggaacgagaccgaccccaagcgcctgggcaccagctctatcgataccacgtgcacgatctgggacatcgagaagggcgtggtggacacgcagctcatcgcgcacgacaaggaggtgtatgacatcgcgtggggcggcgtcggcgtcttcgcgtcggtgtccgccgacggctcggtgcgagtcttcgacttgcgagacaaggagcatagcacgatcatctacgagacgccgtcgccagagacgccgctgctgcgcctggggtggaacaaacaggaccccaggtacatggcgacgatcgtgatggactctaaccgcgtggttgtgctggacatccgcgtgcccaccgtgcctgtcgccgagctgcagcggcaccaggcgtgcgcaaacgcgcttgcctgggcgccgcacagcagctgccacatctgcacagcgggcgacgacgcacaggcgctgatctgggacctcagcgcggtatccaaggagggcgactcgggcctggaccccatcctcgcgtacaatgcaggtcaagaggtaaaccagctgcagtggtcttcgacgcagcccgattgggtggcagtctgctttggcaacaaggcgcagatcctgcgagtgtga

SEQ ID NO: 17 - DNA, 4908 bp, Chlamydomonas reinhardtii
  misc_feature 1..4908: note = WD40 repeat protein
  source 1..4908: mol_type = unassigned DNA
atgagcgcgagcgacaagcgcgagcggcaggaggtttatacatatgtggcaccggacccggtgtacgccatgaactggagcgtgagttgccctcgggcacggctatgacgggcgctcgctgggctgctggggacggggcttgggaaggcgggaggcccggcgccgtctgcagaccctgccgctttgtttgggtggataggactgtgtcgcggcaaacgtccttgcggactgcactggcttgaatgccatgagccaaggggcgctgtggagaggcagagcagagggcggggatgccaagcagcgcgtggcgtggcggccgccgcccctgcctacacctgactgccctcggggctgccgacgattgctgactcgctgtacgacaaattggcgtgccagagggccggggctgtgggatggcgatggcagtaacattctgcaagcatgcctctgctgtgccaccacgtgctcacacacccacactcgcctgcacccgctaacttgcccatgacccacacctacaccccgcacaaccctgcacacccgcacccgcaaccatacctaccacaggtacggcgggacaagcgcttccgactgggcgttgcttcattccgggaggacgtcaccaactatgttgacattgtgtcgcgtgagtccaggcgcacacggggcgattgagtaacatggggattgcggacggcagttacactgttacgggggtatgaagtggagctgtagagtacagccggagataagcacatcgttcggcacagagggagtgtgtcttggccttgcccaccccacactgctccacttccccgcctccccgcctccccgctccccgcctcccccccgccccccgcctgccccagtggacgacgagtcggacgagctgcgggcggaccccgggctgcgcttcccgcacgactacccggccaccaagctcatgtggatgccggaccgcgagggctgtcggcccgacctgttggcgaccacaggtgcggcggggctgtggcggtggtggtggtggtggtggcggcggtcgtggtggtggtggtggcggcggtcagggatgggatgtgggttgggggattgttcgagatgtctggcgcaggtacaagtggccgggctggcagcagtgctgcacttggtaacactgccgttatgtgtctgacacaagccgcacacacgtctgggctggtacatgcacctgcttcggccggtttacacggattacccgccaacacacacacacacacacacacacacacacgcacacacacacgtgcacacacaaccaccacacaggggaggcgctacgcatctggcgtgtgtgtgacggctcggagggcgaggagagcggcagcgggccgggcggacgcggcgttcagctgcggtcactgcttaacaacgtgagcggcgtgcggggcgcgcgtttgcgctgtgtacgctgtcatgttagtgttagggggcacatgggaaggcaaacgggcagggcagtgtgtgcgctgcgtgatctgtgttgcggtggtgtgtgcgggccgtgtgccgtcatgggggctgcgtgtcgcgtgcctgcataggttgtggggttgtgtgtatgtgtgtgtgggcgatcacaatgtggggttcggacccgggtgtgaggagggttgggcgggttgcgcgaactcatcagagcggcagctgcgctcacgctgcaagcccatccaaatctacggtcacaactgtttgcacacccacgaacccttcaaacaaaccgcccaaacccgcatcaacgttctcgcacctcgcagaacaagcagtcggagttctcggcgccactcacgtccttcgactggaacgaggccgaccccaagcgcctgggcacctcctccatcgacaccacctgcaccatctgggacatcgaggtgcggcgcggatggcggcgtgcggcgtgtggcgtgtggcgtgggatggaggctggtggtggtggcggcgtttcgtccgcccttgcttaacgtctcacctccctggagtgacctgcagtcgctcaccgcggctgtgtgcgtcctctgtgccttacgcctccaccgcccccccccatcgcccagcaagccatgccagtacccccccccttagctagtcatgaaggtaaacctcccccgccccacccctcgtcttcggagccctgccaagctcctcccccacctcctcccccgccggcacctgctgtctacttcgctacacttctctgtaccaatccttgcaacccccgccggcaacgacgcggttgctgtgccactgtgcctggctacaacttcacttcttggtttattatgaatgtagtaacccatctccccaccaccccctcctctcccccacctgcagaaaggcgaggtggacacgcagctcatcgcgcacgaccgggaggtgtacgacatagcctggggtgggctgggcgtgttcgccaccgtcagcgcggacggcagcgtgcgcgtgttcgacctgcggtgagcggagggggggcgagagggcgggagggagggagggagggcatcgggatggggggccgggcgggagggaggccttttcatgttatgatcacagttttggagcgctggcgggggatggcgaggtggttcacctaaaagcaaccatgacacacgaagatgtagggcaggcgtgggtgcgcgcggggagggggctgaaaggcgtgcgcctgcgtggtacggggttcgtgtctacggcgcatggggggtcgtggctgctgccaggcctgtcgggcgggtggcacgtggcacgtggcgggtggactgctggtcaaactccgacattcccagtcccagcccatgtccgcacgggtcccgagccactcccatcccgcccttcctcccgccccgcatcccccatctcctcctccccctcgccgcagcgacaaggagcactcgaccatcatctacgagagcccgcagcccgacacgccgctgctgcgcctgggctggaaccggcaggacccccgctacatggccaccatactgcaggtgtgcggcggctgggtatatgtatgtgtgtgtgtgtgtatatgtgtgtgtgtgtgtagcagctggtggcatttgcgcttggcagccattggccgttggcaagacaggcaggaacaattcacgatttggagagcgggaccgttgatgtgatcaggtggcggtttgaagcgaacatcagggtgtgtgggtggggggggggatcagcaacagggctaacgcggcgggcgcctcagtgcggcactgacagctgcacgcggtggcggcaggcgagcgcaaacgtggagcgcaagcgtttgctgcgtcgccagtcgcgcgagtgcattgctgtcactggcggcatggtggtggtgatggcggtatgtgtcatgctggctcagcccctttccctctctccctctccaccacatccgccctttgcgctctgtctttcgtggcccatccatctcctcgcctcgcctgcaggactcgcccaaggtggtcatcctggacatccgctaccccaccctgcccgtggcggagctgtgcaggcaccaggtgcgaggcggcggagcggttgtgtgcagggcggcggcggtgggtgggtgggttggttggtttctagctgccagggttacggcaggggaaggagcaaagacagagcgatggcagtgcgggcgattgtcgattggagccagtcgcggggtctcggagggggcccagcacttgcagttgaaaggcgctgggttttgcgaggtgaaacggattgtgttgtgttattggagttgcgggacctttcatttgctgtgctctgtgccgctgtttcctcacaggcgccggtcaacgccttggcctgggcaccgcactcggcccaacacatctgcaccgccggggacgactcacaggtgtgcacaaggggtggcggaatggggcaggcagcgtgtgtggttgtgtgcggtggttgggggttgcaaggattggtacagagaagtgtagcgaagtagacaacaggcgtggggtggatttccagcgtggcttgtactcgagctcctctccgttggtgtgaccgccaagccaagccgggctgcttccgaagctgttttcgtcagccccatcaccggtgcccggccacctctgcaaagcccgcgcctcgcgctcgccccaacgcaacccttgcctcgcaccccgtctgcccacccacgcacaggcgctgatctgggatgtgtcggcggtgggcggcggcaacaatgccaacgcggcggcgggcggcggcgccagtgacgtcagcttggaccccatactggcgtacggagcggccagcgaggtaaggggggtttctggggactgggtctttggggctggggttctggtgtttgcgtgtgtggggggggggtgtatgtgtgtgaaagaagaaaacgaaacgatgtgtgtgtgtgtgtgtgtgtgtcagagagcaaggaaacacaggagagagtgtgtgtgcatgtgtgtgtgttagaaataacacgagataggtgtgtgtatgtgtgtttgcaagcatgcaccaaacccagccgcgaacccatcctgtcggtgaggtgcgaagggtggagggcgtgggttacagcggtgtagtttgcttcagctggttgagtgcattggaaaggcgtgcgcgtcagaagggctcgcgcgacgagaagaggggtgtgttccgtgggcattgggggtgctgtgggtggtggcgaagaggaggggcggcgccgaagggcctgcggtttgggctgggttcgttgccggtgctgctccggccttgttgcgccccaaccggcatcccccatcacaattgcaggtgaaccagctgcagtggagctccgcgcagccggactgggtggcaatctgcttcggcaacaaaacgcagatcctaagggtgtga

SEQ ID NO: 18 - DNA, 5770 bp, Coccomyxa sp.
  misc_feature 1..5770: note = WD40 repeat protein
  source 1..5770: mol_type = unassigned DNA; note = Coccomyxa subellipsoidea
gcttgagcgcacaccagataagtgccaagttcaccatcgcacgtagcaatttggcagctgccaactgttgaacacccattggtgatcaaggttaggaaaaaccacgaactctgcaggaggatcaaggatcaaatcttgaagtgatgatagttggatctcaatggtagaatggatgggagactgaatgacagacgggcggagatatacacctacgactctgagaacatcgtatatggcttgagctggagtgtaaggatccattcagtctatcttcctgaatttaattgctgcattatgtattgttccacttgcccctgggtcaagtcagtccatgcactcttacttgctacccctctggagacacagactgactggattgttctccttgctgggctgcagaaccgccgtgacaagaagtttcgcctggcagtgggcagcttcatcgaggagtacgacaactacgtcgaaatcatcacacgtgcgtgccgtcctagtctcaccttttgcaccttcacctgcaccgaacctgccctcccctgcttgtgaaactctcctcaaaccacagttgagccacatgcaagaaacagaggaagatccgacaccaccggctgtttattgctcaacttgatgataattacgagatgaatttgcatgagggtaggactggggtgagaggggtagagcactccttggatggcccaaagccataccacttacagaccctcttccaccaatgtgttgcagacaggcttagtgcgcaaggagccagaaactgagatcagatgataccaccaaactgttgaagacagggctttgggtgcgaaggtccaaagcagaggggaaaagatagattcggtgctgcagaaaggcttgcataagctaagattcagacactctcaccaatctgttgcagacagtctatggacgcaagggtgcaaaaattgagatccatatattctttaccagtctgttgcagacaggctttggctgcagaggcccaaagccgaggaagaaagtttggttcgggtgtgtagacaggcttgcagaaccagaggtccacgtactctgaccagtctgttacagacagattcttggggtgcagaggcataaagcagacggggaaagattgactgggttgtccaaacaggctttgtgcaggcaggctctatgcagacaggcctttaagtgcagcttgagaagcagagacccagacactctcaccaagctgctgcagacaggcatgggatgcaagggtccagagcagagctgagagggagaaagcggttcggttttacatgacgcctgcagacagtccgagacaggcttgcagaataatacgattatctgttcgatagcacaacgtgcaaattcatcacagataagctctgggtgcaaagcttcagaagttgaattccagatgctctcacccatgacaggcgtgggtgacaaagctccagaagctgagagggaaagagtggttcggttgtacaggacgcctgcagccagtcttgagacaggctggcagaggctgacagggagacattgtttgtctgcgcaatcgatgacatgacatgcgagttcacatcggacgctcagatctagacaccctcaccaatgcgttgcagacagggctctggatgcaacgctgcagaagctgaggagggctggcattgtctggcccatcagggcttttttgccgtcgtcaattcgccgttagggggagtttatgattgcagcgaggtgtagattttagggttcgattttagggtttaaggtttaaaaccttattcccccgtgacggcgacgacgatgaaaaaagccatatgcctgggctgagcagacgggcttgggttgccaaacttaaaaattcgagaatgaaccaggggtgtgtggtgcgcgcagtcgatgacgcgacatgcaagttcacgtcggacgcgcagctggcgttccagcacccgtacccacccacaaagatcatgttcatgccggacaaggagggggcgcagccggacctgctggcaaccactggcgactacctgcgcatctggcagctgaaggaggacggcacgcagctggtcaagcttctcaacaatgtgcgcctccttccctagtaaaattgtctgtttattagtatcccacgctgttgatatcccccggggagattgcgcagcctcaaacaagcttcaggtaaaggtcgacagctgaaggaggacggcactcagctgatcaagctgctgaacaatgtgcgcccgctctcagaaagacaaatggagaaagaagtatgcagacatcaggcagcgcgagcgctagaagatagaagaaagggctggctcacaagtgcacgtcatgcaacaacaatacaatgatacccagccctgacaattcttgttattgtgaacacatgctgtcagattacccccctcctaccaagggggggaaggtgcggctgcaaacaagcttcaggtgacagatggctgctgaaggaggatggcacgcagctggtcaatctgctcatggatttgcgcctgctccagcatagtgaccaattattgtaatcttcacatgttgtgtgcaattgtgtattttctccttcgcgccgacagggcccgccactgtaggcaggatacggcttacagacgcggtaggaacctactagagatttgtggtggctgcagcttacggtgcctgccatgtagatgacggaacccatggtgtggtggtggtaatgggtttgggccacatgctgtgatgtgctgcccttctgtatcagagcagtgcatctcacttcctggttcaggctgacggattggaaataactgagctcaacatcggaactaagtctttcaatccagaaggcccttttcgtcgatcttcgcacggaagtggaagtgcagccgccccaatatctggttcaagctggactctttcaggcaggactggcaggactgaggcttgcttgcgtagttgagcttgtgcagccgccacccctgtgcgctggaagttagcagtgctgaattgtgtgtggcttatgtgtgaactatctgcgcagaacaagaacagcgagttctgcgcaccgctcacctccttcgactggaacgagaccgacctgaatcggctggggacgtcgagcatcgacaccacgtgcaccatctgggacatcgaggtgactcgctcacacccttgcaccatgatttcaagggtcttgaactacagtaagacacatattttctggcttgcgtaggggccagagcacagtgtcccctgagctgagaatcccctgtgtgactagctttacatggaagcctaaccagatcgcctcatgcagtcctagtcacacaggggactctttgttgtatgatcttattgaaagatcttttctagtctcaaaggggactctttacctaacgtcaaatcagcgcgctctgagtggcatatcagataagggagtgtgtctcaatgtatgtcaccaactggaccctaaggccagtctgacctctaggtggttcctcacatgatagaccggtactgagcatgtcactgagctgagctggcggtcgcgtgattgtctctgcagaaaggcgtggtggacacacagctcatagcgcatgacaaagaggtctatgacatcgcttggggcggcgtgggagtctttgcgtcagtctcggctgacggctctgtgcgcgtatttgacctccggtgcgcccctgaactggcaggtgtcgattgatgagctggcgctggctcattgggcgatattggaagtgtcttagggagttcatttccttgcatagcagtcaggagagtacttgcatggacaatggaactcgatgacaaaggcagccctcccatgttcgccccacaccctactcccacacctgaagcaagaggtgaagtgggacttgtgagggcagaagcacattgtactggtgtttacagaagactacctcttacgggcctgagtgccagctgctgggtctcacaaaagcgccgaaaaaattgcactcctttcccttgctgccaaggtctttcttggcgactgctagagtgacgaagaagtctgtaagcaggtttgaagctccttggcactgtgattgtgatcagcggggggcttgtttgctgatcagcaaggagtgcattgctgcctgcagatatcatgcaggctatgcatccccgccctcctatctggtgccttcatatttgcaggtctacctcaaactttggggtacagtgcatcacattgaggtgccgcttttgcagggacaaggagcacagcacgataatctacgacagcccgcagccggacacgccgctgctgaggctggggtggaacaagcaggacccccgctacatggccacagtcctcatggactccaccaaagtggtcatcctggacatcaggtgacacactgtgcttacgcttctttgaataattgcactgtaacgggcggccctcttctctgcagtttctgaatttctgaatccattacctctctttaaacgctgaatggagtgtactacgctgggtttgcccattatgtcaacattaacaatgaacactgtaattaggaataataatatggctgccagcaaggtggagggcttgccaggcatcttcaggcaccgccctgtgcgcctcggaaaatagaatttcaaacttgaatttggcacttgtgccgcggcaggtaaccgtctaggtccacaccctcctgggatgtcagaccctgcttgagcctcggggactctatttccacctttgtacttttcacccagagggcccagatgagctgtaagtggggcgacccgtcgcatgagcttgaattcaaaagctgtgccgtggcaggtatccgaccctgccggtggcggagttgcagaggcaccaggcgccggtgaacgcggtggcgtgggccccccactcctcgtgccacatctgcagcgcgggggacgatgctcaggcgctgatctgggatctctcttccatgtctcgccccatggaccagaccctgggtgagcccaccttgatctagatcatgtgtatctcatttgctacacgcctgtcctgccaaaagtcaagattaattcttgcagacaagctgcagtaaaaggctgccacaacctacccctcgttggcctcgccaaatgcaagcaagacccaatcaaccgtaggcacggtgctcgagctcagtgtttacaccgccccctgtcagactgggcgtccatgtggcaggaatgctgcaaggagggagctcgtcgggttgatggtgtgtgggaaggagtgggcggaagattgttgtgcttgaggggcatacatgttgtcaaggcatgatgtgctgtgctgcgcagatcccatcctggcatacagcgctggggcggaggtgaatcagctgcaatggtccacgacgcagcctgactgggtggccatctgctttgcaaacaagacccagatcttgagggtgtgaggggattgcgaacctgtgaggcgtttgtctcagtccaattaaatctgtgaaggggtgccagaatggatgtacaggagcgtcgagtagaattctatattcttggtatcgggttacgttcgggttgttcctgttggaacggttgagctgtgttttagttctgcctgtctggcacaaaaaaatgtctgttctggtccccgtggactgtgaatgagagaaccggctgagatctttgagaacctattgacatgaaggttgcagcacattaagaaaaggaggagcgctcttggtaaccttgctgtaacgttgctagatgc

SEQ ID NO: 19 - DNA, 5079 bp, Volvox sp.
  misc_feature 1..5079: note = WD40 repeat protein
  source 1..5079: mol_type = unassigned DNA; note = Volvox carteri
atgagcaacagcgacaagcgtgcggagatttatacctatgtggcccaggaccccgtctatgcgatgaactggagcgtaagtgtcggactttggtgttttttctccctcttcgcaactcgagccactcatagtcactcgcacttaagggtcgtaacgtatcgcctgacgggatggtactggggtcatggggctgacaaccaccgctagctcactttgcattcccgggctgtgggggagggtggtgtgagggcggggaggacggcacaacgggtggcagtgcgtagcccctgtccatgccatagtttaaatgctgcttggagcttgcgttgagctctcgacacccccatgccgccgccttagatgtaagggagcagtagggacgtcggtgagcgggcccctcttaaccccctttccacaacgaaatacacacgcacaccgtgtcggcccggcccagtcactccagcccccatggcttttctctccccgccgacaggtccgccgtgaccggcgctttcggttggcggttggttcgttccgcgaagatgtaaccaactacgtggaaatcatcagccgtgagtacggcagctacgtcggtgatggggtcccgtctctagggtgccacataaggttgccccttttgctaagtgtcgttgttgatactgttgttgttgtggtggtggtaatggtaatggtaatggtaatggtaatggtaatggtgatgatgatggtgggcgtgggtgtgctttgggtgttgcgcatgggaagtgcggcttccctccgcccactctccaacccactctccccttcccctccccccccacacacacactcacacacacgcagtggatgacgatgctgctgagctgcgctccgacccctccctgcgcttccaccacgactaccccgccaccaagctcatgtggcttcccgacagagagggctgcagacccgacctgctagcgaccacgggggaggcgcttcggatctggagagttctggatccggattcagttgcgggggacggggaggacgtggcggcggcgggggcagggggaggaggagcgacgggtgttgggcggggtgtgcagctacgagctctgctgaataacgtgagtttggttttggggaggggggccgccgtgcgtttggctgcgtttggggcgggggacccctgagataggttcgtatcattagctaatatacttaatatcttgaatattcctggattttgcttttacgctagcagaacttgctattgcacgtacaccattattatccattaccgaaccaggaaattaccataccgccaagcaactggtcacagcccttctgccccacccccgggtgcccccgggtccggctcctgctccccccttcccccccgcccacggctcattgaaaccggccacccttcagcggcaactgatatccgatattcctcagagcttatcactgcagatgaaccttcaatttactttttattcctatcgacacgggcgcagacttgttgccctatcccctgtggtagggcccgttctttgactttggcatcatcacgtaactgaggtggccccaggtgaccgacttgactaacattgatgtgccccttcctgtgctggtgctggtgctggtgttggtgttggtgttggtgttgttcagaacaagcagtccgagttctccgctccgctgacctccttcgactggaacgaagcggaccccaagcggctgggaacctcctccatcgataccacctgcacgatctgggacatcgaggtgtgtggagcgtgagggtgggggggccctctggggggctgtgggggttggggggaggggaaggtgggaggggaaacccgctgatcccggggtttgggaccgttggatttgcgcgggacatgggtataggggtttcgggggatcggggtttgagctggggagctgtggcgtgttgggagagagaagggagggaagggtgtgctgagcggtgggtttcgtggggctgatgtgtttttttatgtgagatgtgtgccggtgttgtgggcttatgtttcctcccttttcgaccctctgcacccctctggccttcttccttacacatcacctagatcccgttccccctcctctctcttcgcctctccgcccccatacacacaaccacattgatcgcgtaaagaaaggggaggtggacacgcagctgattgcacatgaccgggaggtgtacgacatcgcctggggaggactgggggtatttgcaaccgtgtcggcagacggctccgtgcgcgtgttcgatttgcggtgggtttggattgggtttggattggctttgtatgtatgtatgtatgtatgtatgtatgtatgtatgtatgtatgtatgtatgtatgtatgtatgtgtctatgtatgtatgtatgtatgtatgtatgtgtctatgtgtgcatgtgtgcatgtgtctatgtgtctatgtgtctatgtgaggtttgggcagtgtggtgagaggcgccatgggcgccgtgggtttcaggacacggtgtctccctgggaccggacctctccccgttgactggccgggactcgttgaccatcagcctgatgtggcgcggccctcgcctcccgccctacttcaaattcaaatttcccgccctccgctgtccggctcctacccccttattgaacgtttcgacagttgattttcctgagctggctcctccttgtatcattagatttcgcttcaccctttcgctctgttccgctctacgccccgcctttgctgcccctcctccccgctccctagtgacaaggagcacagcaccatcatctacgaaagcccccagcccgacacgccgctgctgagactgggctggaaccggcaggaccccaggtacatggccaccatactcatggactcacccaaggtgaggggaagggagagggggagggggccctaagggggagcatggagggcatgcaacgagcaaaacttactggaaattagcatccccagtgcggcagcaaagccgggaggagaggccggaggaaagcctgcagcagcagcagcagcgcgcgtcccaagtaccaaaacattccatatattacgtatacgctttctgcacctgtatgggggtgttgtgtagtgttgtgatttgtgatcgtacgcgcgtatttggtaaccccccctcctgctgtccggcgcaacgccccatcgttgctttgctgcctggacgtgtatattccctctgtcccgtacgttcccgggtgccttgcacgtaggaattccctcccccctcatttccccacccccgtgtgtgtgtgtgcgtgtgaaccccttaccccccaccccccacccccaggtggtaattctggatatccggtaccccacgttgccggtggcggagctgcaccgccaccaggcacccgtcaacgcactggcctgggcgccccactcagcacaacacatatgtaccgcgggggacgactcgcaggttgggaggaggggggcgggacccgtgggtcggtgcagggaaggatggcgccacggatgggaacacggaagggccgatattatttgctgttgatgctacatttgcaggtgacatatggcccgagttggcgttttgccggtgtgtgtgtgtgtgtgtgtgcgtgtgtgttatccccgtatgtgctgctgcacgctgtactgtactgccccgggtgctcgtcgaggcggtcgatccctcggcacgcatcctcgcggtttcttgcatatgattcccagttcccctccccgtccccctccccgtctcgctcgcaggcgctcatctgggacgtctcagctgtgggcagtggcggtggtcagccgggggcgttaggggggggaaccgcgggggatgtgtccctggatcccatcctggcgtacggcgcacagagcgaggtacggcagagcagaggggatgaaactagaagtgggtggtcaggaggatggagctaatgaacagactgtgcggaagggttctggagttcggaatgaatcagctggtactgggtagtagctggttccgatgtgggtaccaggtggtggtcggttcggcaggtgggcgacagcggtgtcgtggggcgccgtggaacacatccagctatcttccccggttaatgagggcttgggtcgcattttctttggcggggtttcatccacagtgattggattctttggaggagtgcaggccaggtagcgcgctctgcatgatgtgaatgaacagtgtggtgcagcgcagctctgttgcaccacccacagaaagctgcgtcgagcctcggcgcgaatacacgtgtgggctaccaccattgccctgcattgcagtccggggatgtgggacgctcatgcagagaaaaaagctgaaccgcctttctccccaatcggccgctgggggacgcatgcagctgatgatgctgccgtcctgctgccggcgttgtctgtcccaccaacacgatctgcccccttccgtaaccctgtctgtctgtctgtccgcccttctatcctgatgttttgggctgccttttccttgcaggtgaaccagctgcagtggagctccgcgcagcccgactgggtggccatatgcttcgccaacaagacccagatcctcagagtgtgacccctgtgggacctgcggcgacttggctaggcttggataactcagcggctgcgactttgcccatgtttcgcactcccgtagtgcagatggcacgagtgtgggggcaggcaggatcaacagcgtcgccttgcctcaggccacatgactccgccatgtctgggtgtgtaggtccgcgggcgctgcataaaaaaagcaatcgtggcattggagagcgcggatgcgtgccgaaaaatcttgcgttttgcccgagggctgatgggtcctgggtcgaggaggaagggtgcggttagagaggactttcacactgaatggctttctttgaggagccctcgacccctgtatgcaaatttagcctccccgtccacaaggaggcatgctgcggctttttgcggcaagctgtccaaaatgtggcatgcgtattccagaggagttttggatgtaagcaggtggaacctggg

SEQ ID NO: 20 - DNA, 3272 bp, Chlorella sp.
  misc_feature 1..3272: note = WD40 repeat protein
  source 1..3272: mol_type = unassigned DNA; note = Chlorella variabilis
gtgaactcggccggttgccatccaggagagagccaacccctgagcccctgcacgggtgaggaccgtcggaggaaggcgcagaatgccggcgcggggcgccagagcgggggagtgcgggcgccgaccgccgcccagcagcagcagcgccggccgcttagcagcagcgccttgtggggggtcgtgcgcttatctcctcgcattctctcttacaggctcccatgcaggaccagcagcagcagggggaggggcgggcagagatctacacctacagcagctcggccagcgtctacgcctgcggcttctcggtgagcagccagccaatggcagcggccgccgtcagctcttccagcccagagtgcagcacaggcagcgcaggcagcgcagcacgaagtctcagagcatgtcggccgctgcgctgcagccgtgtgcccggcagcttaccgccactctcgccgctcccgccgctgcgtgcctcccttgcagtcccgccccgacaagcccttccgcctggctgtcggcagcttcatcgacgactatgcaaacaaagtggagatcatccagtgtgagtgtctgtttttaggcaggcgctgcgctgcacggactgcggatggcacggtgctgccgcggctgctgtgattgcacgcggctgctgctcgggtctggcgccgccgctggggtgggatcttgtctggctctggctctggatcttcatctggggcgcagtcttgggcatgtgggtggtagcacgctgctcagaccaggccccacctgccgccaccaccactgcctatgcgcgccacccgcagtggacgaggcggccggggtggtgcgcaacaaccctgcgctgaccttccagcacccctacccgcccaccaaggtcgccttcatcccggataaggtgaggcgccgaggccgggctgactgagccaggttgtgggctgcctgccgggacaaggcagcagcctgcgggtgctgtcctgcccctgcccctgacctgaccgtgctgccccgtatgctggctctgtgatccccgccttctgaacgccaccacctgccgccgccgcctcctgccccagagcgggacccgccccgacctgctggccactagcggcgacttcctgcgcctgtggcgtgtgtcggatgagcccggggcgcagcagggcgtgcgcctggagaagctgctgaacaatgtgagggcgggcgggcgggcggctgcgctgcagggggtgctcagcgagggtgggagcaggggtaggggcgcctcggtgcggcgagtgccggcagggctgctggggagcgacgtgccggccagtgtacagagcgcaccctcgccgccagccctcgacgcggcgctcgcaccacctcacaccctgccgtctccctctctcccccatgctcccatgctcccatgctccccttcccgcccgccacccgccgcagaacaagggcggcgactttgcggcgccgctgacgtcctttgactggaacgagctggaccctcgccgcgtgggcaccgcctcgatcgacacgacgtgcacggtgtgggacgtggagcgcggggtggtggacacgcagctcatcgcgcacgacaaggaggtgggtgggtgcggcgcggggggtgtagtgtggccacgggcgtggcgtgcctgcttgtgggtggagcatggggtggtggtcaggccgcagttgattgcgagctacaagggggtgggtggggtggtggcttctctaggcggtgctggtcctgtgttcagccatgcgcttgagcatatgtgcacaccgattgcatggaggggtgtgggcgtggtggggcatcaacatcacacctgcctgcccctgcccgcccctcaaagacctcgctgccgcccgcccgcctgcccgcctcccccacacacatctatgccaggtgtacgacattgcctggggcggcgtcgggatctttgcctcggtctccgccgacggctcggtgcgcgtgtttgacctcaggtgggaacagccggctcacccgccagccgggctgcgtgcccgcctgcccagccgcttgccctccggcgcagcccgtgcacacttctgcgagctccgccgccaggccgcaaacccacgcacgctcgtggccccgccagaaacccacgcacgcacgctcacccgcttgcgcatgcatgcacttattgttgtttgcccacagggacaaggagcactccaccatcatttatgagtccccccagcccgacacccccctgctgcgcctgtcctggaacaagcaggacccgcgctacatcgcaggtgcggcgcggccggcgcggggcctcgcggtaccatcgtgccccagccgagctgccttgcggctgctcaggcccaggcacacaggcactcaagcacaccaccacaaccaccacagccaccactttcacacccacgcacacacaagccgttccttccctgcgtgcagtgttagccatggattcgccgcgggtgacggtactggacatccggtaccccacgctccccgtggccgagctgcagcggcaccaggcgggggtcaacgccatctgctgggccccccacagcgccacccacctgtgctccgcgggcgacgacagccaggcgctgatctgggacctgggcctgctgggcacgctggggcagcagcccgagggcggcccgccgggcgccgcggcggcgggtggcggcctggacccgatcctggcctacaacgcaggcgccgaggtcaaccagctgcagtggagccccgcccagccggactgggtggcaatctgcttcggcaacaagacccagctgctgcgggtgtgaggcgcgtgccgacagagcaccacaccgccgcgctgctcgccggctgcagcgttgctcgccgctcccctccagggcagcgcggcgcccgcgccgttcctgcttcccaagctgccagccttcttgcgtcccttattcgcctgctccccctgtcttgcttccgctcctgctgtactgccccccgcccgcatctgtatcacccgggtgccttttcttcactgcacacgtaccaccgcatctgtggcaccctgcccctccctaatgcacggccctctggcacgctgccagcccctctaatgcatgggccctgccattcacaccaatcgcatcaaccgtacatctgtccccctgcactgctctttgtcaccattccacctgatgctctctcctctcccaacccaactgatccccccgtcgcatccataccgatttcgagagacaccttgcaatgaaacacgcagcgc

SEQ ID NO: 21 - DNA, 3032 bp, unidentified
  misc_feature 1..3032: note = WD40 repeat protein
  source 1..3032: mol_type = unassigned DNA; note = Parachlorella sp.
atgcagcgcgcagaaatccatacgtacgagagccccacgttggtttatgcactcaactggagtgtaagtcacacaatatgttgacaagatactgaagcgcgacatgtataggctgtgtcctgaagaggcaagctttcacctgctgtaggtgcggcctgacaaacccttcagactagccatcggaagctacatcgaggactacaacaaccgagtggaaatcgtcacacgtatgttcaccgcttcaggtcttgctgctattgcgttcgtgccttcaaagtcagactttcgaagcaagtttgcccaccgggggtctctggtgcagtcggtgaagatggaaatggaatgcggcctagcccacggcacacctttcagcatccctatccacccaccaaacttcagtttgtaccagatcctgatggctcccggcctgatctgttagccagctccggtgacttccttcgactctggcgcatcacggaggacggggtttccctggaaaagcttctcaacaacgtgagcgcgcctgctctgatagcgctgtcctgttgtaccatggacggctagcgcacagcggtagcgcgcagtgcaacgaagacgaccggggctgacgctactctgaatgcaaccaccttgcctgctgtggtgcagaacaaagcaagcgagttttgcgcgcccttgaccagctttgactggaatgagaacgaccccaagagggtgggcactgccagcatcgacaccacctgcactgtatgggacatagagaagggggtggtggacacccaggtgggttgagtggagtagagtggagtggcggagaacctggaagagcaggccatgaccagggcatgcgtgctgagcactcctgccgccgccgccgtcgcgtcaagtcttggccgttcaaagactcgaggtccttccggtgtgctgcttggagtcgcccgcgtcctgccacatcgtgtgcctgctgccgctttggagccatgctccccttgccacgcgctcacccttggaaccgtgtggcccctgcagctgattgcccatgacaaggaggtgtacgacatagcatgggggggcgtgggggtgtttgcctcggtgtcagcagatggctcggtgcgggtgtttgacttgaggtaggtgctctgcctgcggccttcgacctggggctgagtgaccgggtggggccggcctggaggatggagggtggaatatggtgcagtacgcgaggtacatgatcttggcggtggctcatcctggtgtgggtcgctggtagaagaagcggcgtggaacaggtgtaggtgtggggcttggagcagtgaggtgcaagccagttgtagagtatgccccagcagcctccccaaagggccctcagcccgcagcaccctgccagcaggagcttccccagttgctgcgccaggggcagcgcatgctcgggcgcacagctgtgtgtgtgcacgacagagccagctcctggtgtggagctggcggtgggcacgtgtgggagggctctgttggggtcccatgcgttagacagggcatggcaggggtctgggcgtgtgggcaggtcgtgcttctgcctcatgcgccctccacccaccctcaccccgctgctgcctcatacgccccgcacccaccccctccccccttcctgccgctgcctcatgcgccctccaccaacattccctgcagggataaggagcactcgaccatcatctatgagtcgccgcagcccgacacccccctgctgcgcctggcctggaacaagcaggacccgcggtacatggccaccactgccctcaactcctccgccattgtggtgctggacatccgattccccacggtgccggtggtggagctgtccaagcaccaggtgcgatatggcagcgggggtgcagtcgctgcggggggtgcctgcaatggggcagctgctatctgccgccttgctgtgagctggcctggcgctgggagccaggagtgctcaccctgctgcgagcgctgccacgggttggctgcatcgcacaggctgaggcccccgggctgggctggaagggcgggtgcctcaccggctcaggcgcccacgaggatcgccgatttcctgccctggcctggtgatcatgccgctggcggcggtgcggtggcgtgcgcagcatgcggctgcgcactgcattggccagccacgctgctcgtgccatggtgtgcatgcagcccgcaccagccaaagcgtgctcgcgctgcccgctgctgaacagacctctcaacgcgctccctccctctgtggctagtgtgccccagcaccaggtggcagctggagcgcgcccagcatggtgcagcaacaggaggaaagccgccgcccctgggcctgcgggcaattttagcgcgttgcgctgcgctgcattcctcgcaacgtgcacgtcaccgaagcgtgtatctccccccttccccactgcctgcaggccgcctgcaatgctgtggcctgggccccccaaagtgccaaccacatctgcagtgccggggacgattgccaggtggggcgcgctggaaggggggaaggaaagggggaggggggtcctgtgaactgggcagaatgctggcctttctaataaaccgccaccaacaagctggggctgtgcttggggagggcgggggcagtcgtcccccgcggcacgggggccgacacccttgccttcccttggcgagtattcgagcacgggaccccttcctcccctcctgtgcatctcatgtgatgtactcgccgagccctctgcccctgctgcgactgtcgcgcagctgcaaggccacgcgccaagcgcattgcggcgctgtccctccgcgcctctgtgccgtgcgtgcaggctttgatctgggacctgtccactctgggggagggcggcgcgggccaggcggggagcccccccctggaccccatcctgtcctacatggcgggggcggaggtgaaccagctgcagtggtcggcgtcccaccccgactgggtggccatctgctttgggaacaagacgcagattttgagggtgtga

SEQ ID NO: 22 - DNA, 1045 bp, unidentified
  misc_feature 1..1045: note = WD40 repeat protein
  source 1..1045: mol_type = unassigned DNA; note = Picochlorum sp.
atgtcgactgattggttggttgtaattgtgcagtctaggcaagataagggtgtacgagtagctgttggcagttttgtggagggtgtctcgaacacagtcgagattcttcgcggtacggatcatctgtgagtctctgttctacttgggaaatgtgggcataattttttgtatatgaggtgcagtgactcccgcaggtttaattgtggatgacaaggaaacgttcggcatagagtatccggcgacgcaggtgggatttattcctgataggttttgcaacaagccagatttgttggcaacctctggggatgctgttaggttgtggaaaatttcagacgcagggacgacgcttgaactggtgttgaatgatccaaagaatacctctaaaaatttcagtgcggtgacctgctttgattggagtgaaatcaatgtgaaagtgttggcggcagggtcgagtgcagggcgattattgctgtgggacaccgagtcagggaggctgcagggcacaatggtgggacatgaggatgagattcttgattgtcagtgggcagctaatgatgtgattgtttcttcttcgggcgatggatcaattcggatgtatgacctgcgggataaagaccattgcacggtattgtatgagacccccaggaggacccctgtgccgaggttttgttggaacaagctggatccgaggcatcttgcattttctatagaaaagagtcggcttgttagtgttctcgatgttcgctttccgacagagccggtgatcttgctggacggtcatatggggaactgtacagcacttggttggtcccctcacagagaggaatacctctgctcggttggagatgattgccatgcattgatatgggacgtggggaaggtgaatagtgaggaggatagtaagccaaatcgagaggcggtggacgcatctcctatcctagcgtacaatgctcaggcagagatcaatgcgatggcttggaatccaatagacccagattggattgccatttgcgctagaaacagaacacaagtattgagaatatga

SEQ ID NO: 23 - DNA, 2857 bp, Tetraselmis sp.
  misc_feature 1..2857: note = WD40 repeat protein
  source 1..2857: mol_type = unassigned DNA
atgccgacgagcgacgcgacgcaggcacatgagcaccatcacgtgccgcacgcgcggccgcgacccccgccaagaacgttgtctgcatgtgccatgttgatgagcaaggagaaacggcggcgccacgcagacaagagctggccgattttttggccagtgtgtgtgtggctagctttcgcgagctccaccaagaaagcctcagcgtgcgggccagctcgctttgcataatgcttcggcaatttgattgttgactagccgctacagtacatgtacaacggagcgtcgcctacgggtatacggagggcttgccgtaactgtggatcgctgcctattaaacggccgtcgtaaagatagacgctgcaagggtttcactggtgtcagtcctgtcccgggggcgggaaaaccaaaaccctggtcgtaaatgagacgtggcaaagtttcaccggcgccagtcgcgtatccaggaaaaaaaaacccgccccctcgcactcttcgtggccacaggtagaggactgccgaagtgatcaccccacagcaaagcccaccgtcgtcagcgctacccgttacgtgcgggtactcagactctcgccgcgacacccacgcaacaagccaacaacgccgccccgcttgcggatcgcttcacgcttgggctggtgcggaaccttgttcagggaccttgttcccgccgacaaattattgctgctcggctctcctgcactccgacagcgtggcaccaggctggcctgttcaccgcccggggccggtgtgggttgtggcccacgcgagtggccactcgggttcctttcagcctacgctgggcgtaaagcccttaccagcatgcttgtcctgcacgcgccccgtgctgccacgggctgacgcactgatcctgccgtgttgcgcttgtggcggcaggctcggtgctgtgccccgggcgacgcgcgcctgggtgggctagcttgcgatggcgagcgggccggaggaccggggtgcgggggcggcgggggcggcgccccaccaacgaggcgatagcaacggcaaagcggtgacagacaagcgcggggagatatacacctacgaggcgccgtacccggtatacgggatgaactggagtgtgcgtgcgccggacatggccaaggggggccaggggaggcccccccgggggggggggggggggaggagttacttggtacaagcagactttggccccgtggctgagggggtcgagtgttgcaggtgcgggaggatatgaagttccgcctcgcggtgggaagctttgtagaggacgtggagaatgcggtggagctcatccggcgtgagtgcccgagcgcgtacgcaggcctgcctgctgtgtgcagggagcgcgcgggcatccgcctgatgacgtgctgtgtgcacagtgaacgaggaaaccggcaagtttgagagcaacccggcgcacaagtttgtgcacccgtatccacccaccaaaataatgtttatccccgaccgcgactgctcgcgccctgacctgctcgccaccacgggcgattatctgcggctgtggcgcgtggaggaggacggcgtcacgctgcacaagctgctgacaaatgtgagcgcggacaaacctttgtgcccgccccccccccaccaccaccagtcctccttccctctaaggcccattctcaagagataccacggccagctccagcatgacccccgcccccctatttcgtcaacatgcacctcccccctgcagaacaaaaacagtgagttttgcgcgccattaacatcctttgactggaacgaagccgacccgaggcaactgggcacctcatccatcgacacaacatgcacaatatgggacatcgaggtgggccaggcagtccaagccccccccccccccccccccccgcaaatgccctccttgcgctgacatgtcaaaacgcctgcctcgtggtgcgtccagagaggcgttgttgacacgcagcttatcgctcacgacaaggaggtgtacgacattgcgtggggcgggcagggcgtctttgccagcgtgtccgcagacggctctgtgcgagtgtttgacctccggtgcgccctgcggtacctgcggccgcgcgcgaccccacctgggcgccgtggctggcttgcgggggggggggggggggggctgacgcgccggcgggctgttcggcaccgcagggacaaggaccactcaaccatcatctacgagagcgggatgcccgagatcccgctgctgcggctgggctggaacaagcaggacccgcgctacatggccaccatcctcatggactcctccaaggtggtcgtcctggacatcaggtccgccgcccttgcctgccatcacgcaacatatactgggggtgtgtctggcggcgctgacctgtgatgctgcgccgccgtgcctgctgcaggtaccccacgatgcctgtagctgagctggaggcgcatcacaagcctgtgaatgccctggcgtgggccccgcagtcctcctcgcacatctgcactgcgggggacgacgcacaggtgtggcgggatgcatgctctgatgcatcacaggagacagagcgacagggggggggtgagcagggggggggggggggggaaggggggatgcccgggtgaggcagatggtggctgactgttgcatgctgccgcccaggcgctcatctggaaccttgcgcccatgggcacccaggggcccatggggggtgctgcgcctgcagttctaggcgcggacctggatcccatcctagcgtacaacgccggcgaggaaatcaatcagctgcagtggtccagcacgcagtccgactgggtgggcatatcctttggcaacaagatccagattttgagaatctag

SEQ ID NO: 24 - DNA, 1431 bp, unidentified
  misc_feature 1..1431: note = WD40 repeat protein
  source 1..1431: mol_type = unassigned DNA; note = Ostreococcus sp.
cggtcgacgcgctcgacgcggtcgacgcgagcgaggcgcgcgcttcgagcgacgcgaaggcgtcggacgcgaacgcgaaggcgtcgagcgcgaacgcgggacccgacgggcggtgaaggcgcgcgacgaaagacgaggaaggcgcgcgcgaacgatgaacgcggagaagagggcggaaatatacacctatgaggcgccgtggatgatctacgcgtgcaattggagcgtgcgtggcgagcgaggcgatggattgggggcgagcgcgggagaattgaatcgcgaggggcgacggaggagacgcgacggaggagactcggggacgcgcgcgaacggtcgatcggagattaaaaatggagacgcgcgagtgaagacgcgaatggcgtggactgacgacgtcgaattgaacgcgacaggttcgacaagataaacgcttccgcctcgccttgggttcgttcgtggaggagtatagcaacaaggttgagatcatcaccttggacgaggaaaccggggagtttccgaaggaggcgcagtgttcgttcacgcatccgtatccttgcacgaaaattttgttcattccggacaaggagtgcacgaaggaggatttgttagcgacgacgggggactacttgcgaatctggcaagtgcaggatgataacacggtgcagatgaaatctttactgaataataacaagagcagcgaattttgcgcaccgctgacgagctttgattggaacgagaccaagcttcagcgagtggggacgtcgtcgatcgacacgacgtgtacgatttgggacatcgagcgcgagtgcgtggacacgcagctcatcgcgcatgataaggaggtgtacgacatcgcgtggggtggtccagaggttttcgctagcgtaagtgcggatggaagtgtgcgagttttcgacttgagagacaaggatcacagtacgatcatttacgagagtcaaactccagacacgccgctgctgcgtttggggtggaacaagcaggatccgagatacatggccaccatttgcatggatagtccggtgatcattctcgatattcgcttcccgacgttgccggtcgcagaacttcagagtcacagagcgagcgtgaatacattggcgtgggcgccacacagctcaagccacatgtgcacggcgggcgacgacagtcaggcgttgatttgggatttgtcgtccatgaatcaaccacccgaaggcggtctcgaccctattctcgcttactctgctggagcagaaatcaatcagttacagtggagcgcgtcgcaaccggattggatctcgatagctttccgaaacagcctccagatcctccgagtttagtcaacgcgctgtcaggtctgcgccgacgccactgtatattacccgaatttccggatacgcgacacacgacacacgacacgcacgcacgtag

SEQ ID NO: 25 - DNA, 1294 bp, Micromonas sp.
  misc_feature 1..1294: note = WD40 repeat protein
  source 1..1294: mol_type = unassigned DNA; note = Micromonas commoda
atggcggccatgggcagcggccagagcggcgccgagatttacacgtacgaggcgccatggctcgtgtacgcgatgaattggagtgtgagtgcccgtcgatgactctgctccgtcccgccgcgttcctccccgcccgggccgatccctcgcctgcacccaatctgacccggcaagatccgctgctgacgcgactttgaggacgcgcccggcagtcgaccgacgcgccccgccccgcgacgtgacccgctgacgcttcactcgatataaacctccccctccccgcgcgcgcattcaacaggtgaggcaggacaagaggttccgcctcgcgctcgggtcgttcgtggaggagtacagcaacaaggttgagatcatcacgctggacgagcagcgacgggagtttccggcggagccgacgcacaggttcgaccacccgtacccgtgcacgaagatcatgttcgtcccagacgccgagggaaccagcgaggacttactggccacgagcggcgactatctgcgggtttggcgcataggcgacgacggcgtgcacctgcggagcctcctgaacaacaacaagaacagcgacttttgcgcgccgctcacgtcgttcgactggagcaccaccaacctggcgagggtgggcaccagcagtttggacaccacgtgcaccatctgggacctggagaaggagacggttgactcgcagctcatcgcgcacgacaaggaggtgtacgacatcgcgtggggcgggccggaggttttcgcgagcgtctccgccgacgggagcgtcagggtgttcgacctgcgggataaggaccacagcacgatcgtctacgagtccccgacgccggacacgccgctgctgaggttgggttggaacaagcagaacccgaggtacatggcgacgatggagatggacagcgccaaggttgtggtgctggacattcgcgtgcccgcgctgccggtggcggagctgaagaagcacagagccgcggtgaacacgctggcgtgggcgccgcacagctcgaggaacatatgcaccgccggggacgacgcgcaggcgctcatttgggacctgtcgtcggtggcgcagcccggggaggacgggatggatccgatgctggcgtacaacgcgggggcggagatcagtcagctgcagtggagcgcgacgcaaaccgactggatagccatcgcattcggcaaaaacctgcaggtgcttcacgtgtgacgcccgcggggagaacgtggcgatcgtagtcctagttcggttttgaattcaacgttcatttagcactca

SEQ ID NO: 26 - DNA, 1041 bp, Arabidopsis sp.
  misc_feature 1..1041: note = WD40 repeat protein
  source 1..1041: mol_type = unassigned DNA
atgggaacgagcagcgatccgattcaagatggttccgatgagcagcagaagcgatcagagatctatacatacgaagcgccatggcacatctacgcaatgaattggagcgttcgtcgcgataagaagtatcgtctcgccatcactagcctcctcgagcaatacccgaaccgtgtcgagattgtgcagctcgatgaatccaatggtgagatccgttccgatcctaacctctcctttgagcatccttatccaccaacgaagaccattttcatacctgacaaggaatgccaaagacctgatcttctcgctacttcaagtgatttccttcgtttatggagaatcgctgatgatcattcccgtgttgagctcaaatcttgtctcaatagcaataagaacagtgagttttgtggtcctcttacttcttttgattggaatgaagctgagccacgtcgaattggaacatctagtactgatacgacttgtactatctgggacattgagcgtgaagctgttgatactcagcttattgctcatgataaggaagtttttgatattgcttggggtggtgttggtgtttttgcatctgtttcagctgatggctccgttagggtgtttgatcttcgtgataaggaacattcgacgattatctatgagagctccgagcctgatactcctttagtgcgtcttggttggaacaaacaggatcctaggtacatggctactattatcatggacagtgctaaagttgtggtgcttgacattcgttttccggctcttcctgtggttgagcttcaacgacatcaagctagtgtcaatgccattgcttgggctcctcatagctcttgtcacatttgtactgctggagatgattctcaagctttgatttgggatatttcatccatgggacagcatgttgaaggtggtcttgaccctattctagcttacactgctggtgctgagattgagcagcttcagtggtcctcttctcagcctgattgggtcgcaattgctttctctactaagctgcaaattctcagggtttga